(19) United States
(12) Patent Application Publication
(10) Pub. No.: US 2019/0367656 A1
(43) Pub. Date: Dec. 5, 2019

(54) MODIFIED ALGINATE COPOLYMER, ALGINATE NANOPARTICLE AND APPLICATIONS THEREOF

(71) Applicant: Agency for Science, Technology and Research, Singapore (SG)

(72) Inventor: Jatin Kumar, Singapore (SG)

(21) Appl. No.: 16/479,583

(22) PCT Filed: Jan. 22, 2018
(86) PCT No.: PCT/SG2018/050037

§ 371 Date: Jul. 19, 2019

(30) Foreign Application Priority Data
Jan. 20, 2017 (SG) 10201700470R

(51) Int. Cl. C08F 251/00; C08B 37/00; A61P 35/00; A61K 31/047; A61K 9/51; C08F 299/02; A61K 31/704; A61K 31/337; A61K 33/243

(52) U.S. Cl. CPC C08F 251/00; C08B 37/0084; A61P 35/00; A61K 31/047; A61K 9/5161; C08F 2438/03; C08F 299/024; A61K 31/704; A61K 31/337; A61K 33/243; A61K 9/5138

(57) ABSTRACT

There is provided a modified alginate copolymer comprising an alginate backbone and having a grafted moiety attached to one of the hydroxyl groups of the alginate backbone, the grafted moiety comprising a polymer and a stabilizing group, the stabilizing group comprising at least 2 heteroatoms independently selected from the group consisting of N, S, P and Si. In a preferred embodiment, poyl(ethylene glycol)methyl ether methacrylate is grafted onto alginate via macroRAFT polymerization or ‘click’ Chemistry approach. There is provided a drug delivery method using the cation-mediated self-assembly of modified alginate copolymer as defined herein. There is provided a process to make the modified alginate copolymer as defined herein. There are further provided cosmetic applications and medical applications of the modified alginate copolymer as defined herein.

### CROSS-REFERENCE TO RELATED APPLICATION

This application claims the benefit of priority of Singapore patent application No. 10201700470R, filed Jan. 20, 2017, the content of which is incorporated herein by reference in its entirety for all purposes.

### TECHNICAL FIELD

Various embodiments relate to a modified alginate copolymer, a nanoparticle formed therefrom, a process for making the modified alginate copolymer and the nanoparticle, and the application of the alginate nanoparticle as a drug delivery system.

### BACKGROUND

Alginate is a naturally abundant anionic biopolymer derived from algal and bacterial sources that is composed of blocks of consecutive and alternating (1,4)-linked-b-D-mannuronate (M) and a-L-guluronate (G) residues. It is widely used in healthcare, cosmetics and food because it is non-toxic, cheap, biodegradable and highly sustainable. Alginate poses a distinct characteristic in that it cross-links in the presence of divalent cations (Ca²⁺, Mg²⁺, Ba²⁺) and can form acid gels at low pH (FIG. 1a).

The cross-linking property of alginate is attractive for its use as an encapsulation material. It has been demonstrated as a suitable carrier for food and supplements, proteins and enzymes, cosmetics, cells and drugs. Alginate has also been identified as a good candidate for gene delivery and tissue engineering. To obtain alginate nanoparticles for encapsulation, the present strategies involve the use of surfactants in order to chemically stabilize alginate during crosslinking. However, this technique is hindered by poor reproducibility and scalability, which renders the use of cross-linked alginate to very basic applications due to difficulty in processability. Moreover, this cross-linking is often undirected and the morphology of the cross-linked monomer is highly dependent on the method of cation introduction—a physically driven mechanism.

Modification of alginates thus far have resulted in the loss of this unique cross-linking ability of alginate and thus the polymer is exploited merely for its low-toxicity, sustainability, water solubility and biodegradability. It has been shown to be effective in transfection leading to stimulation of growth factors or in cancer therapy.

Cross-linking mediated encapsulation with alginate is performed either by dropping an alginate solution into a calcium bath, introducing calcium to alginate that is stabilized by a surfactant, microfluidic techniques, performing a directed self-assembly or cross-linking on modified alginate (FIG. 1b). The first two techniques are hindered by poor reproducibility and scalability in the nano or sub-micron scale.

Moreover, there are also no examples of calcium mediated self-assembly, as this almost always relies on far more sophisticated methodologies. For example, Meng et al. demonstrated the formation of hollow nanospheres of alginate-graft-poly-(ethylene glycol) via an associative host—guest interaction of the grafted poly(ethylene glycol) chains with a-cyclodextrin. Vegas et al. demonstrated the modification of alginate with cell binding motifs and polymers, such that these modified alginate polymers self-assembled onto the surface of mesenchymal stem cells via a polymer—cell interaction. However, to the best of the inventors' knowledge, no examples of spontaneous, calcium mediated self-assembly into polymeric nanoparticles has been reported in the literature thus far.

The literature related to the preparation of alginate as an encapsulation material showed firstly that a driving force is required in order to constrict the cross-linked alginate particles into a particulate morphology. Secondly, it is noted that the functionalization of alginate is often performed on the carboxylic moieties, thereby restricting its ability to cross-link with calcium ions. While there have been several examples of the functionalization of the hydroxyl moieties on alginate, these techniques often result in the opening of the ring structure on the backbone. Moreover, the lack of solubility of both sodium alginate and alginic acid in organic solvents significantly limits the modification of alginate to ones that can be performed in aqueous solvent systems, usually on the carboxylic acid moieties. An example of an exception to this is by Grøndahl and co-workers, who demonstrated the functionalization of the hydroxyl moieties on the alginate backbone in an aqueous medium while preserving the ability to cross-link in the presence of calcium ions.

Schleeh et al. and Pawar et al. demonstrated the modification of alginate so as to allow for its dissolution in organic solvents. The sodium cation was replaced by tetrabutylammonium (TBA) cations to afford polymer solubility in dimethyl sulfoxide (DMSO), dimethyl acetamide (DMAc) and N-dimethyl formamide (DMF) with 1% tetrabutylammoniumfluoride (TBAF). This development was followed up by Kapishon et al., who then functionalized the hydroxyl moieties of TBA—alginate with a bromoisobutyryl initiator upon which single electron transfer—living radical polymerization (SET-LRP) was performed to afford poly(methyl methacrylate) (PMMA) grafted alginate, with a good degree of control over grafted chain length and PDI. A polymerization induced self-assembly was observed for the amphiphillic copolymer into micelles of around 50 nm, with a PMMA core and an alginate shell. However, no studies were undertaken to demonstrate calcium mediated polymer behaviour.

Therefore, there is a need for an alginate, which is easily processable, easily scalable, and which is capable of forming nanoparticles.

### SUMMARY

In a first aspect, a modified alginate copolymer is provided. The modified alginate copolymer comprises an alginate backbone and has a grafted moiety attached to one of the hydroxyl groups of the alginate backbone, the grafted moiety comprising a polymer and a stabilizing group, the stabilizing group comprising at least 2 heteroatoms independently selected from the group consisting of N, S, P and Si.

This present body of work demonstrates a method through which alginate can be modified and grafted with polymers so as to not only retain its cross-linking characteristic, but also to spontaneously self-assemble into nanoparticles upon the introduction of Ca²⁺. Advantageously, the grafted polymer moieties act as stabilizers that force the modified alginate copolymers into a nano-assembly.

In a second aspect, a drug delivery method is provided. The method comprises dissolving the modified alginate copolymer as described above in a first solvent, and subjecting the solution to an M²⁺-containing source, subsequently collecting the solid of the ensuing reaction mixture and redispersing the obtained solid in a second solvent.

Advantageously, the modified alginate copolymer allows for spontaneous self-assembly into nanoparticles upon the introduction of a divalent metal. This provides for the ability to encapsulate active molecules and a sustained release over time is demonstrated.

In a third aspect, a process for making a modified alginate copolymer is provided. The process comprises subjecting alginate to an acid to obtain alginic acid, subjecting alginic acid to an alkylammonium solution to obtain an alginate-alkylammonium-salt, grafting a moiety on the alginate backbone, and polymerizing the grafted moiety with a polymerizable moiety, wherein one of the grafted moiety or the polymerizable moiety comprises a stabilizing group, the stabilizing group comprising at least 2 heteroatoms independently selected from the group consisting of N, S, P and Si.

Advantageously, by functionalization of alginate with various moieties, including, but not limited to RAFT agents, alkynes, polymers, thiols, it is possible to perform graft polymerization of various polymers to and from the alginate backbone.

In a fourth aspect, an alginate nanoparticle comprising a modified alginate copolymer as described above and M²⁺ is provided.

In a fifth aspect, a dental hygiene composition comprising the alginate nanoparticle as described above is provided.

In a sixth aspect, a cosmetic composition comprising the alginate nanoparticle as described above is provided.

In a seventh aspect, a cosmetic method of improving dental hygiene is provided. The method comprises administering to a mammal an effective amount of the alginate nanoparticle as described above or the dental hygiene composition as described above.

In an eighth aspect, a cosmetic method of improving skin complexion is provided. The method comprises administering to a mammal an effective amount of an alginate nanoparticle as described above or the cosmetic composition as described above.

According to a ninth aspect, an alginate nanoparticle as described above for use in therapy is provided.

According to a tenth aspect, use of an alginate nanoparticle as described above in the manufacture of a medicament for the treatment of cancer is provided.

### BRIEF DESCRIPTION OF THE DRAWINGS

The invention will be better understood with reference to the detailed description when considered in conjunction with the non-limiting examples and the accompanying drawings, in which:

FIG. 1 shows the difficulty to synthesize alginate nanoparticles for encapsulation as natural alginate cross-links upon the addition of Ca²⁺ and alginate in solution will form a hydrogel with Ca²⁺.

FIG. 2 is a schematic showing of the grafting of polymers onto an alginate backbone, and its calcium mediated self-assembly into a nanoparticle (FIG. 2a); in one example, an alginate-graft-POEGMA comb polymer was utilized (FIG. 2b)

FIG. 3 shows the FT-IR spectra of the PPEGMEMA-N₃, alginate-alkyne and alginate-click-PPEGMEMA for the HMW (FIG. 3a) and LMW (FIG. 3b) samples. The FT-IR shows the R—N₃ stretch at 1970 nm for the PPEGMEMA-N₃, which disappears completely once reacted with the alginate-alkyne, denoting the complete reaction of azides.

FIG. 4 shows the Z-average diameter recorded for (a) 2 mg/mL; (b) 1 mg/mL; (c) 0.5 mg/mL; (d) 0.25 mg/mL; (e) 0.125 mg/mL HMW alginate-graft-PPEGMEMA solutions as a function of CaCl₂ concentration, and (f) the particle size and the sample PDI at 1M CaCl₂ for the different solutions.

FIG. 5 shows the TEM image of 1 mg/mL HMW alginate-graft-PPEGMEMA in 1M CaCl₂ solution showing 200 nm particles.

FIG. 6 shows the Z-average diameter recorded for (a) 4 mg/mL; (b) 2 mg/mL; (c) 1 mg/mL; (d) 0.5 mg/mL; (e) 0.25 mg/mL LMW alginate-graft-PPEGMEMA solutions as a function of CaCl₂ concentration, and (f) the particle size and the sample PDI at 1M CaCl₂ for the different solutions.

FIG. 7 shows the Z-average diameter and the associated PDI of a 2M solution of CaCl₂ as a function of the concentration of (a) HMW alginate-graft-PPEGMEMA; (b) LMW alginate-graft-PPEGMEMA.

FIG. 8 shows the Z-average diameter and the associated PDI of 2 mg/mL solution of (a) HMW alginate-graft-PPEGMEMA; or (b) LMW alginate-graft-PPEGMEMA titrated against HCl up until a pH of 0.

FIG. 9 shows the concentration over time of the released 4BR (FIG. 9a); percentage of 4BR released over time as a function of the total concentration released (FIG. 9b).

FIG. 10 shows the concentration and the percentage of 4BR released over time for 1:1 HMW alginate-graft-PPEGMEMA (FIG. 10a); 1:8 HMW alginate-graft-PPEGMEMA (FIG. 10b); 1:1 LMW alginate-graft-PPEGMEMA (FIG. 10c); 1:8 LMW alginate-graft-PPEGMEMA (FIG. 10d).

FIG. 11 shows stacked ¹H NMR spectra of LMW-alginate at its 3 stages of functionalization: (a) alginate—TBA; (b) alginate-BM1430; (c) alginate-graft-POEGMA.

FIG. 12 shows first-order-kinetic rate plots of the polymerization of OEGMA (65° C.) with the two different macroRAFT agents, HMW-alginate BM1430 and LMW-alginate BM1430 (x=conversion).

FIG. 13 shows the Z-Average diameter recorded for (a) 2 mg mL⁻¹; (b) 1 mg mL⁻¹; (c) 0.5 mg mL⁻¹; (d) 0.25 mg mL⁻¹; (e) 0.125 mg mL⁻¹ HMW-alginate-graft-POEGMA solutions as a function of CaCl₂ concentration, and (f) the particle size and the sample PDI at 1 M CaCl₂ for the different solutions.

FIG. 14 shows the Z-Average diameter recorded for (a) 4 mg mL⁻¹; (b) 2 mg mL⁻¹; (c) 1 mg mL⁻¹; (d) 0.5 mg mL⁻¹; (e) 0.25 mg mL⁻¹ LMW-alginate-graft-POEGMA solutions as a function of CaCl₂ concentration, and (f) the particle size and the sample PDI at 1 M CaCl₂ for the different solutions.

FIG. 15 shows the Z-Average diameter and the associated PDI with a 1 M solution of CaCl₂ as a function of the concentration of (a) HMW-alginate-graft-POEGMA; (b) LMW-alginate-graft-POEGMA.

FIG. 16 shows the TEM image of self-assembled HMW (top left)- and LMW (top right)-alginate-graft-POEGMA, and the respective images of 4-n-butylresorcinol encapsulated particles at a polymer to 4BR ratio of 1:1 (HMW: bottom left, LMW: bottom right). Scale bar at the bottom left of each image for reference.

FIG. 17 shows the concentration of 4BR released over time for (clockwise from top left) 1:1 HMW-alginate-graft-POEGMA; 1:8 HMW-alginate-graft-POEGMA; 1:8 LMW-alginate-graft-POEGMA; 1:1 LMW-alginate-graft-POEGMA.

FIG. 18 shows the molecular weight (closed symbols) and polydispersity index (open symbols) development with conversion of the polymerization of OEGMA for the (a) HMW alginate macroRAFT agent and (b) LMW alginate macroRAFT agent.

FIG. 19 shows the Z-average diameter recorded for 2 mg/mL of each polymer in methanol as a function of CaCl₂ concentration.

FIG. 20 shows a TEM image of: (FIG. 20a) HMW; (FIG. 20b) LMW alginate-graft-POEGMA loaded with doxorubicin.

FIG. 21 shows release curves of doxorubicin from HMW and LMW alginate-graft-POEGMA.

FIG. 22 shows release curves of paclitaxel from HMW and LMW alginate-graft-POEGMA.

FIG. 23 shows fluorescent microscopy images of pig intestine that had been washed with: (left) a fluorescent dye; (right) a solution of fluorescent labeled alginate-graft-POEGMA nanoparticles.

FIG. 24 shows the particle size of the HMW (FIG. 24a) and LMW(FIG. 24b) alginate-graft-PEG with respect to CaCl₂.

FIG. 25 shows the TEM images of the cisplatin cross-linked HMW (FIG. 25a) and LMW (FIG. 25b) nanoparticles.

FIG. 26 shows the Cisplatin release curves for the HMW (FIG. 26a) and LMW (FIG. 26b) loaded alginate-graft-PEG nanoparticles. The series are numbered according to cisplatin to COOH ratios (1, 0.75, 0.5, 0.25).

### DETAILED DESCRIPTION

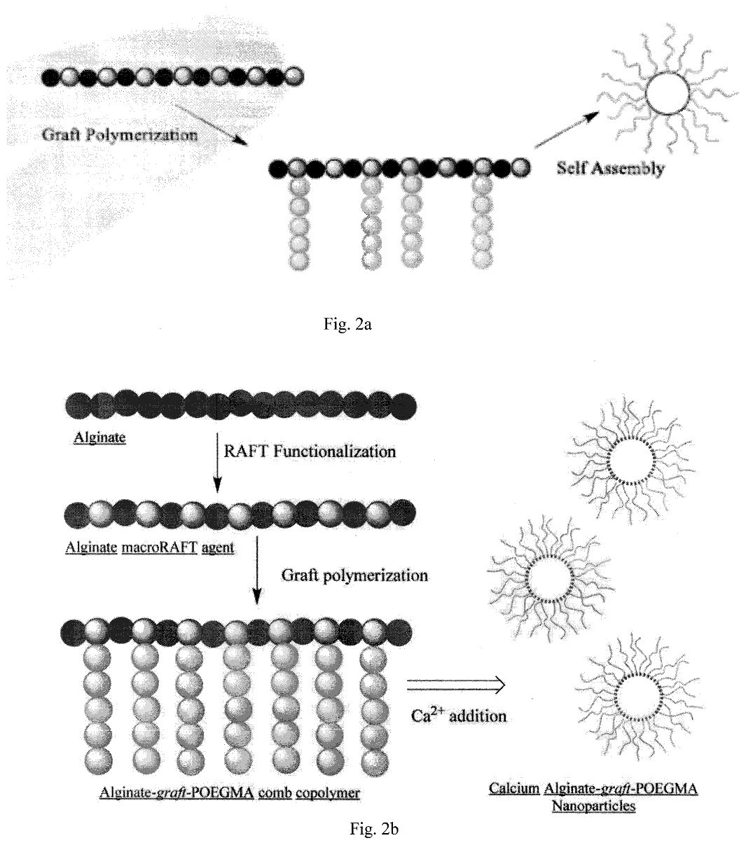

Various embodiments refer to a modified alginate copolymer comprising an alginate backbone and having a grafted moiety attached to one of the hydroxyl groups of the alginate. The term “modified alginate copolymer” as used herein refers to a copolymer which is derived from an alginate backbone, having a functionalized moiety attached to it, which is polymerized. The structure of this copolymer may be described as a “comb” copolymer, as shown in FIG. 2. Alginate based comb copolymers were synthesized by reversible addition—fragmentation chain transfer (RAFT). Alginate was used both in a high molecular weight form and a depolymerized, low molecular weight form and prepared into a macroRAFT agent by solubility modification with ammonium ions and functionalization with a RAFT agent on its hydroxyl moieties. A polymerizable moiety was then polymerized on the functionalized moiety. The copolymers dissolved well in a range of organic solvents and demonstrated self-assembly into nanoparticles upon the introduction of calcium chloride in both aqueous and methanolic solutions with particle sizes ranging between 100 and 500 nm Remarkable encapsulation efficiencies of a bioactive agent was demonstrated and a sustained release profile was observed in aqueous acidic media. These new materials complement a growing library of biodegradable and sustainable polymers that show notable potential for the use in encapsulation and drug delivery.

With the above in mind, various embodiments refer in a first aspect to a modified alginate copolymer comprising an alginate backbone and having a grafted moiety attached to one of the hydroxyl groups of the alginate backbone, the grafted moiety comprising a polymer and a stabilizing group, the stabilizing group comprising at least 2 heteroatoms independently selected from the group consisting of N, S, P and Si.

The “stabilizing group” may also be termed as the functionalized moiety. This group may be utilized for both the polymerization of the polymerizable moiety on the alginate backbone, as well as introducing stability for the encapsulation of bioactive agents into the modified algiante copolyme, in order to form the aginate nanoparticle.

The grafted moiety on the alginate backbone may comprise hydrophilic polymers, such as OEG, hydroacryalamide, acrylic acid, 2-(dimethylamino)ethyl methacrylate (DMAEMA) or polysaccarides. The directed self-assembly of the modified alginate copolymer may be the result of the polymer and the stabilizing group.

In various embodiments, the polymer which is poymerized on the grafted moiety may be an acrylate-based polymer. Hence, the monomer which is used for the polymerisation may comprise an acrylic acid moiety, which consists of a vinyl group and a carboxylic acid terminus. The acrylate-based polymer may be selected from the group consiting of methacrylate, ethyl acrylate, 2-chloroethylvinyl ether, 2-ethylhexyl acrylate, hydroxyethyl methacrylate, butyl acrylate and butyl methacrylate.

In preferred embodiments, the the acrylate-based polymer may be a methacrylate-based polymer.

In various embodiments, the polymer may additionally comprise an oligo(ethylene glycol) moiety. The term “oligo(ethylene glycol)” may be used interchangeably with “poly(ethylene glycol)”. The oligo(ethylene glycol) (OEG) moiety may already be polymerized at the time of attachment to the alginate backbone. The OEG moiety may have a molecular weight of about 100 to about 500 Da, or about 200 to about 400 Da or about 300 Da. Typically, the oligo (ethylene glycol) moiety consists of about 5 to about 1000, or about 100 to about 1000, or about 500 to about 1000, or about 100 to about 800, or about 100 to about 500 repeating units of ethylene glycol.

In various embodiments the poly(ethylene glycol) moiety or the oligo(ethylene glycol) moiety may be attached to the oxygen atom of a carbonyl ester. The carbonyl ester to which the PEG or OEG moiety is attached to may be the carboxylic acid of the acrylate moiety. Hence, in a preferred embodiment, the grafted moiety may comprise both an ethylene glycol oligomer or polymer and an acrylate-based polymer. For example, the grafted moiety may comprise PPEGMA or POEGMA. The OEGMA monomer may consist of 5-50 repeating units of ethylene glycol, and POEGMA may consist of 5-1000 repeating units of OEGMA.

In various embodiments, the polymer may comprise a poly(ethylene glycol) (PEG) moiety, either in conjunction with an acrylate-based polymer (for example, as a PPEGMA moiety) or without an acrylate-based polymer, hence, instead of POEGMA or PPEGMA. In these embodiments, the poly(ethylene glycol) moiety may already be polymerized at the time of attachment to the alginate backbone. The poly(ethylene glycol) moiety may be attached to the alginate backbone by a ‘linker’, which may have the formula —(C(O)—(CH₂)ₙ—C(O))—, which is covalently linked to one of the hydroxy groups of the alginate backbone. In this embodiment, the poly(ethylene glycol) moiety may have a molecular weight of about 1000 to about 10000 Da, or about 1000 to about 8000 Da, or about 1000 to about 6000 Da, or about 3000 to about 10000 Da, or about 4000 to about 8000 Da, or about 3000 to about 6000 Da,or about 5000 Da.

In various embodiments, the grafted moiety may be attached to one of the hydroxyl groups of the alginate backbone by way of a carbonyl ester bond. The attachment of the grafted moiety on the hydroxyl moiety of the alginate may be advantageous, as the carboxyl moiety of the aliginic acid or the carboxylate of the sodium alginate remains unsubstituted and can therefore interact with the M²⁺ ions, which may be advantageous for the formation of the nanoparticle.

In various embodiments, the stabilizing group may be a functional group selected from thiocarbonates, azides and 5 or 6-membered heterocycles.

In various embodiments, the thiocarbonate may be selected from a trithiocarbonate. Advantageously, the trithiocarbonate may be facilitating the RAFT polymerisation. Alternatively, thiocarbonates may be reduced to thiols so as to react with alkynes and alkenes via an addition reaction. This is known as thiol-yne and thiol-ene click chemistry respectively.

In various embodiments, the 5 or 6-membered heterocycle may be selected from an azole or azoline. In one embodiment, an alkyne may be attached to the alginate backbone, which is reacted with an azide to give an azole using a copper-mediated click reaction.

In various embodiments, the azole may be selected from the group consisting of pyrazole, triazole, imidazole, 1-pyrazoline, 2-pyrazoline, 3-pyrazoline, 1, 2, 3-thiadiazole, 1, 2, 4-thiadiazole, 1, 2, 5-thiadiazole, 1, 3, 4-thiadiazole, 1, 4, 2-dithiazole, 1, 2, 5-thiadiazole, 1, 3, 4-thiadiazole and 1, 4, 2-dithiazole.

In various embodiments, the alginate backbone may have a molecular weight between 1 kDa and 1000 kDa, or between 1 kDa and 500 kDa, or between 1 kDa and 300 kDa, or between 100 kDa and 1000 kDa, or between 200 kDa and 500 kDa, or between 100 kDa and 300 kDa. Embodiments wherein alginate with this molecular weight is used, may be referred to as high molecular weight (HMW) alginate, and it would refer to using the alginate in its original form.

In various embodiments, the alginate backbone has been modified to have a molecular weight between 200 Da and 200 kDa, or between 200 Da and 100 kDa, or between 500 Da and 200 kDa, or between 1 kDa and 200 kDa, or between 10 kDa and 200 kDa, or between 50 kDa and 200 kDa, or between 50 kDa and 100 kDa. In this embodiment, the alginate has been depolymerized before further processing. Embodiments wherein alginate with this molecular weight is used, may be referred to as low molecular weight (LMW) alginate This may be advantageous in improving solubility.

According to a second aspect, there is provided a drug delivery method, the method comprising dissolving the modified alginate copolymer as described above in a solvent, and subjecting the solution to a M²⁺-containing source, subsequently collecting the solid of the ensuing reaction mixture and redispersing the obtained solid in a second solvent.

The M²⁺ may be any divalent metal. For example, the divalent metal ion may be selected from the alkaline earth metals. Alternatively, it may be selected from any divalent transition metal, such as from the Groups 10 to 12 of the periodic system, preferably from the Group 10 of the periodic system, for example from Pt²⁺. In one example, the divalent metal ion is Ca²⁺.

In another aspect, a modified alginate copolymer may comprise an alginate backbone and may have a grafted moiety attached to one of the hydroxyl groups of the alginate backbone, the grafted moiety comprising a polymer attached to a —(C(O)—(CH₂)ₙ—C(O))— moiety, which is covalently linked to one of the hydroxy groups of the alginate backbone (this is shown in one example in Scheme 5, Examples section). Without being limited to this embodiment, the modified alginate copolymer according to this aspect may be suitable for forming an alginate nanoparticle with cis-platin (exemplary shown in Scheme 6 in the Example section). In preferred embodiments of this aspect, the number of alkyl groups (“n”) may be between 1 and 10, or between 2 and 8, or about 2. In various embodiments, the polymer may be selected from a polyethyleneglycol (PEG) or a polypropyleneglycol (PPG), or a mixture of the two (for example a poloxamer). In a preferred embodiment, the polymer which is grafted on the alginate backbone is a PEG-polymer. In this aspect, it is shown that the cis-platin may conjugate to the carboxylate functionality of the alginate backbone, thereby crosslinking the alginate, which may result in the alginate nanoparticle being formed. The person skilled in the art would be able to appreciate that the same mechanism may be observed when crosslinking the modified alginate from the first aspect with cis-platin and the PEG moiety may have the same function as the POEGMA moiety in the modified alginate of the first aspect.

Advantageously, the modified alginate copolymer self-assembles upon the addition of M²⁺ which is due to the grafted polymer moieties. In one embodiment, the self-assembly allows for encapsulation of Ca²⁺-ions, which may be useful in dental hygiene, for example, by incorporation of the self-assembled modified alginate copolymer (nanoparticle) into mouthwash. In another embodiment, the self-assembly may be useful in encapsulating bioactive agents. The nanoparticle may have a core-shell morphology, wherein the alginate backbone forms a core and the grafted polymer moieties may be extending from the core as a shell (FIG. 2 a and b).

Hence, in various embodiments, the method may further comprise subjecting the aqueous solution to a bioactive agent before subjection to the M²⁺-containing source. The bioactive agent may be entrapped in the nanoparticle, and may be slowly released. The “bioactive agent” may be a compound that has an effect on a living organism, tissue /cell. In the field of nutrition bioactive compounds are distinguished from essential nutrients. While nutrients are essential to the sustainability of a body, a bioactive agent may not be essential since the body can function properly without them, or because nutrients fulfil the same function. Bioactive agents can have an influence on health. They may be found in both plant and animal products or may be synthetically produced. Examples of plant bioactive agents are carotenoids and polyphenols (from fruits and vegetables), or phytosterols (from oils). Examples in animal products are fatty acids, found in milk and fish. In other embodiments, the bioactive agent may be a pharmaceutically active agent. A pharmaceutically active agent may be, for example, a nucleic acid, including DNA, a peptide, a protein, a small molecule, a cell, an antibody, an antigen, a ligand, a hormone, a growth factor, a cell signalling molecule, a cytokine, an enzyme inhibitor, an antibiotic, a chemotherapeutic agent, an anti-inflammatory agent, or an analgesic. In some embodiments, the pharmaceutially active agent comprises an anti-tumor drug. The anti-tumor drug may be selected from the group consisting of doxorubicin, paclitaxel, gemcitabine, SN-38, trimetrexate, vinblastine and cisplatin, preferably doxorubicin, paclitaxel and cisplatin. Some examples of bioactive compounds are flavonoids, caffeine, carotenoids, carnitine, choline, coenzyme Q, creatine, dithiolthiones, phytosterols, polysaccharides, phytoestrogens, glucosinolates, polyphenols, anthocyanins, prebiotics, taurine, hyaluronic acid and 4-n-butyl-resorcinol.

Advantageously, the bioactive agent may be incorporated into the alginate nanoparticle. The alginate nanoparticle may then be used for the controlled release of the pharmaceutically active or bioactive agents.

In various embodiments, the modified alginate copolymer may be dissolved in a first solvent. This first solvent may be selected from polar, protic solvents, such as alcohols, nitromethane, water, and a combination thereof. They are often used to dissolve salts. In general, these solvents have high dielectric constants and high polarity. In one embodiment, the polar, protic solvent solvent may be selected from an alcohol. In one example, the first solvent is methanol.

In various embodiments, the M²⁺-containing source may be an MCl₂ source, optionally comprising an electron-donating ligand, for example CaCl₂ or Pt(NH₃)₂Cl₂. The electron-donating ligand may be an amine, for example NH₃. Pt(NH₃)₂Cl₂ may comprise “cis-platin” (cis-[Pt(NH₃)₂(Cl)₂]). The MCl₂ may be present as an aqueous solution. The solution may have molarity of about 0.1 M to about 10 M, or about 0.5 M to about 5 M, or about 0.8 M to about 2 M, or about 1 M.

The ratio of the polymer to the bioactive agent may be about 0.1:50 to about 50:0.1, or about 0.5:20 to about 10:0.5, or about 0.5:10 to about 5:0.5, or about 1:8 to about 2:1.

In various embodiments, the obtained solid may be an alginate nanoparticle. The alginate nanoparticle may advantageously encapsulate a bioactive agent or Ca²⁺. The solid may be collected by centrifugation or filtration of the reaction mixture.

In various embodiments, the obtained solid may be redispersed in a second solvent. The second solvent may be a biocomaptible solvent. Hence, it may be non-toxic. Additionally, it may be a carrier solvent for liquids commonly used on the body, such as water or paraffin, glycerin, lanolin alcohol, or panthenol.

According to a third aspect, there is provided a process for making a modified alginate copolymer comprising subjecting alginate to an acid to obtain alginic acid, subjecting alginic acid to an alkylammonium solution to obtain an aginate-alkylammonium-salt, grafting a moiety on the alginate backbone, and polymerizing the grafted moiety with a polymerizable moiety, wherein one of the grafted moiety or the polymerizable moiety may comprise a stabilizing group, the stabilizing group comprising at least 2 heteroatoms independently selected from the group consisting of N, S, P and Si.

The conversion from the alginate to the alginic acid may be performed by addition of an acid. The acid may be a mineral acid. In one example, the acid is HCl.

In various embodiments, the process is preceded by a step of depolymerizing the alginate to give a low molecular weight alginate.

In various embodiments, the polymerizable moiety may be an acrylate. In preferred embodiments, the acrylate may be a methacrylate.

In various embodiments, the polymerizable moiety may comprise an oligo(ethylene glycol) moiety. In various embodiments, the grafted moiety may be attached to one of the hydroxyl groups of the alginate backbone by way of a carbonyl ester bond.

Advantageously, the carboxyl moiety of the aliginic acid or the carboxylate of the sodium alginate may remain unsubstituted in this process step.

While there have been numerous publications on alginate grafted polymers, there have not been any examples of reversible addition—fragmentation chain transfer (RAFT) polymerization from an alginate backbone thus far. RAFT is a well-established controlled radical polymerization technique that has been thoroughly exploited for the synthesis of polymers with controlled topology and functionality, which can provide access to the synthesis of functional polymers that could afford stimuli response and biological response.

Hence, in various embodiments, the polymerization may be a RAFT polymerization. The functionalizing group on the grafted moiety providing for the RAFT polymerisation may be a a trithiocarbonate. The process step of the RAFT polymerisation may be carried out under elevated temperature. The reaction temperature used in the RAFT polymerization of the process disclosed herein ranges from 40° C. to 200° C., or from 50° C. to 150° C., or from 60° C. to 100° C., or about 70° C. The RAFT polymerization may further comprise a radical initiator, which may be AIBN. The solvent may be selected from polar aprotic solvents, such as tetrahydrofuran, ethyl acetate, acetone, dimethylformamide, acetonitrile, dimethyl sulfoxide, nitromethane or propylene carbonate. In one example, the solvent is dimethyl sulfoxide.

In alternative embodiments, the polymerisation may be a click reaction. The embodiments referring to the click reaction provide a polymer which is substituted with an azide, which reacts with an alkyne, that is grafted on the alginate backbone in order to form a triazole.

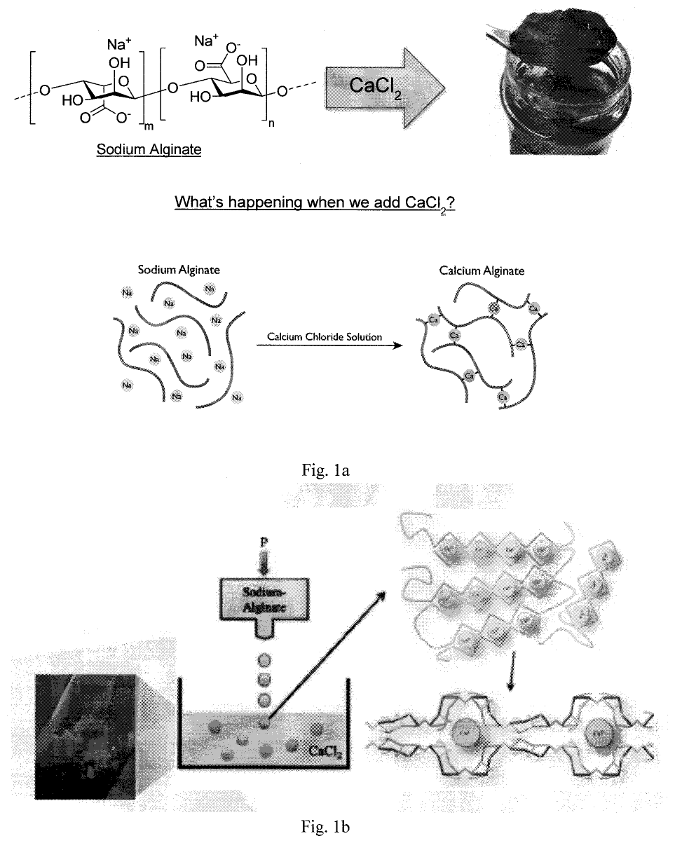

According to a fourth aspect, there may be provided an alginate nanoparticle comprising a modified alginate copolymer as described above and Ca²⁺. Advantageously, by adding Ca²⁺ to the modified alginate copolymer, the modified alginate copolymer does not form a gel (as shown in FIG. 1), but forms a nanoparticle, which is capable of encapsulating bioactive agents. Hence, the modified alginate copolymer may be modified in such a way as to allow for nanoparticle formation, which is particularly advantagoues in a drig delivery system. This may be due to the stabilizing moiety defined above.

Hence, according to various embodiments, the alginate nanoparticle may further comprise a bioactive agent.

In various embodiments, the nanoparticle encapsulates the M²⁺and optionally the bioactive agent.

According to a fifth aspect, there may be provided a dental hygiene composition comprising the alginate nanoparticle as described above. The dental hygiene composition may advantageously provide a sustained release for M²⁺, for example of Ca²⁺. This composition may be, for example, a mouth wash.

According to a sixth aspect, there may be provided a cosmetic composition comprising the alginate nanoparticle as described above, wherein the alginate nanoparticle comprises a bioactive agent. The cosmetic composition may advantageously provide a sustained release for a cosmetic agent, as for example, 4-n-butyl-resorcinol.

According to a seventh aspect, there may be provided a cosmetic method of improving dental hygiene comprising administering to a mammal an effective amount of the alginate nanoparticle as described above or the dental hygiene composition as described above.

According to an eighth aspect, there may be provided a cosmetic method of improving skin complexion comprising administering to a mammal an effective amount of an alginate nanoparticle as described above or the cosmetic composition as described above.

According to a ninth aspect, there may be provided an alginate nanoparticle as described above for use in therapy.

According to a tenth aspect, there may be provided use of an alginate nanoparticle as described above in the manufacture of a medicament for the treatment of cancer.

In various embodiments, the use of an alginate nanoparticle as described above in the manufacture of a medicament for the treatment of cancer comprises using an alginate nanoparticle wherein the alginate nanoparticle may further comprise a bioactive agent, optionally selected from an anti-tumor drug.

According to an eleventh aspect, there may be provided a method of treating cancer comprising administering to a mammal an effective amount of an alginate nanoparticle as described above.

In various embodiments, the mammal may be a human.

Alginate was used both in its supplied (213 kDa) and depolymerized (73 kDa) forms and prepared into a macroRAFT agent by solubility modification with tetrabutyl ammonium ions and functionalization with a RAFT agent on its hydroxyl moieties. Poly(oligo ethylene glycol methacrylate) (POEGMA) was then polymerized from the macroRAFT agents in organic solvent demonstrating pseudo first-order kinetics. The copolymers dissolved well in a range of organic solvents and demonstrated self-assembly into nanoparticles upon the introduction of calcium chloride in both aqueous and methanolic solutions with particle sizes ranging between 100 and 500 nm. Remarkable encapsulation efficiencies of 4-n-butylresorcinol, a lipophillic active pharmaceutical ingredient, were demonstrated in methanol, and a sustained release profile was observed over 6 hours in aqueous acidic media. These new materials complement a growing library of biodegradable and sustainable polymers that show notable potential for the use in encapsulation and drug delivery.

Herein, there is presented a novel method for the preparation of alginate grafted copolymers. The polymers were synthesized using reversible addition—fragmentation chain transfer (RAFT) polymerization from an alginate backbone, which afforded copolymers that self-assembled into nanoparticles in the presence of calcium ions. Alginate was used as received, along with a second variant of depolymerized, lower molecular weight alginate that was prepared by the method outlined by Kapishon et al. so as to circumvent the poor solubility and processability of the larger polymer. The performance of both polymers was compared. Both variants of alginate were modified to alginate—TBA by the method outlined by Pawar et al. to afford them good solubility in a 2% solution of TBAF in anhydrous DMSO. A commercially available RAFT agent with an acid moiety was then esterified to the hydroxyl group on the alginate backbone to afford RAFT functionalized alginate, which allowed for polymer grafting via the polymerization of poly(oligo ethylene glycol methacrylate) (POEGMA). The POEGMA grafted brushes on the comb copolymer stabilized the calcium cross-linked alginate, thereby resulting in the self-assembly of the comb copolymer network into nanoparticles, and this body of work sought out to also elucidate whether the different sizes of alginate backbone had any effect on the self-assembly characteristics. The entire process is outlined in FIG. 2a.

The stabilizing grafted polymers also afforded solubility in numerous solvents, thereby extending the use of alginate in both organic and aqueous media. To assess the potential of this material in the encapsulation and controlled release of therapeutics and active molecules, its performance was demonstrated with 4-n-butylresorcinol (4BR), a dermatological drug for the treatment of melasma. The use of 4BR in dermatological formulations is particularly challenging due to its poor water solubility and its tendency to cause skin irritation, and its limitations could be circumvented with encapsulation and sustained release mechanisms.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

As used herein, the term “about”, in the context of molecular weight ranges, typically means +/−10% of the stated value, more typically +/−5% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### Experimental Section

Various embodiments relate to a modified alginate copolymer, to an encapsulation method using the modified alginate copolymer and to a process to make the modified alginate copolymer.

Exemplary, the RAFT agent, 2-(((dodecylthio)carbonothioyl)thio)propanoic acid, (product code: BM1430) was purchased from Boron Molecular and used without purification. Oligo ethylene glycol methyl ether methacrylate of Mₙ=300 Da (OEGMA) was purchased from Sigma-Aldrich and destabilized by passing it over a column of basic alumina 2,2-Azobisisobutyronitrile (AIBN), (Fluka 98%) was recrystallised twice from methanol. 4-n-Butylresorcinol was purchased from Kumar Organic Products Ltd. All other chemicals were purchased from Sigma-Aldrich and used as supplied unless otherwise stated.

Nuclear magnetic resonance (NMR). ¹H NMR spectra were recorded on a Bruker (400 MHz) spectrometer with CDCl₃ or D₂O used as solvents.

Size exclusion chromatography (SEC). Gel filtration chromatography (GFC) to determine molecular weight distributions of the grafted polymer chains was performed in 0.01 M NaNO₃ buffer on a Tosoh EcoSEC GPC System equipped with two SuperMultipore PW-M (separation range 500-1 000 000 Da) in series heated to 40° C. and a dual flow RI detector calibrated with poly(ethylene oxide) standards. Both single detection gel permeation chromatography (GPC) via refractive index and triple detection gel permeation chromatography (T-GPC) to determine absolute molecular weights were performed in THF on a Viscotek GPC Max module equipped with two Phenogel columns (103 and 105 Å) (size: 300×7.80 mm) in series heated to 40° C. with a Viscotek TDA 305 triple detector calibrated with poly(methyl methacrylate) standards.

Dynamic light scattering (DLS) & static light scattering (SLS). DLS for the determination of particle size and SLS for the determination of molecular mass of the polymers in an aqueous medium were conducted using a Malvern Instruments Zetasizer Nano ZS instrument equipped with a 4 mV He—Ne laser operating at λ=633 nm, an avalanche photodiode detector with high quantum efficiency, and an ALV/LSE-5003 multiple tau digital correlator electronics system. All DLS studies were performed with a sample concentration of 2 mg mL⁻¹ and at 25° C. unless otherwise stated. All SLS studies were performed at 25° C. with a toluene standard for polymer concentration at a range of 0.125-5 mg mL⁻¹.

Transmission electron microscopy (TEM). The sizes and morphologies of the polymers were observed using a high resolution transmission electron microscope (Philips CM300 FEGTEM) at an accelerating voltage of 300 kV. The polymers were dispersed in water (1 mg mL⁻¹) and deposited onto 200 mesh formvar copper grids, and analysed unstained.

4-n-Butylresorcinol encapsulation and release assays. Calibration curves were obtained for 4-n-butylresorcinol in methanol, 20% methanol in 1 M CaCl₂, 1 M CaCl₂ and acetic acid buffer at peak absorbances of 281 nm, 290 nm, 279 nm, and 279 nm, respectively. Acetic acid buffer was prepared at a pH of 5 by mixing 11.8 mL of 0.1 M acetic acid and 28.2 mL of 0.1 M sodium acetate. For the release studies, nanoparticles of a total of 25 mg of alginate and the corresponding amounts of 4-n-butylresorcinol were prepared and isolated by centrifugation, then introduced into 5.5 mL of either 1 M CaCl₂ or acetic acid buffer. The 1 M CaCl₂ solutions were left at room temperature while the samples in acetic acid buffer were incubated at 37° C. From these, 0.5 mL of sample was removed at regular intervals then centrifuged. The absorbance of the supernatant was then measured to determine the concentration of 4-n-butylresorcinol released into the solution.

### EXAMPLE 1

### Depolymerization of Alginate

The method outlined by Kapishon et al was used to depolymerize alginate. Briefly, 7.5 g of alginate was dissolved in 500 mL of deionized water and placed in a large round-bottom flask. To this, 5 mL of pyridine and 3.5 g of ascorbic acid was added, the flask was sealed, heated at 80° C. and sparged with nitrogen. To this, 50 mL of hydrogen peroxide (30%) was added. The solution was removed from heat after 90 minutes and precipitated with cold methanol. The precipitated solid was further washed with methanol and dried under vacuum. The degraded polymer was characterized by GPC and static light scattering (dn/dc: 0.161) to determine the molecular weights, which were: Mₙ^{SLS}:95.8 kDa and Mₙ^{GFC}:73 kDa (Ð:2.43) (relative to PEO standards). The undegraded polymer molecular weights were: Mₙ^{SLS}:277.8 kDa and Mₙ^{GFC}:213 kDa (Ð:3.31) (relative to PEO standards).

### EXAMPLE 2

### Preparation of Tetrabutylammonium Alginate (TBA-Alginate)

Solubility modification for both the depolymerized alginate of lower molecular weight (LMW-alginate) and the untreated alginate of higher molecular weight (HMW-alginate) was performed by adding each of the sodium alginates (1 g) in separate 100 mL solutions of a 1:1 v/v mixture of ethanol and 0.6M HCl in water. The mixture was allowed to stir overnight after which the alginic acid precipitates were filtered and washed three times with ethanol and acetone, then dried under vacuum. The dried products were then dispersed in water (3% w/v), and a solution of tetrabutylammonium hydroxide is added dropwise until all the solids dissolved and the pH of the solution reached 9. The solutions were then freeze dried and tested for their solubility in DMSO, DMF and THF with and without 2% tetrabutylammonium fluoride (TBAF), they were found to be only insoluble in THF, both with and without TBAF. The products were characterized by ¹H NMR in D₂O. ¹H NMR (400 MHz, D₂O, δ, ppm); 1.43 (12H, t, NCH₂CH₂CH₂CH₃ (TBA)); 1.86 (8H, m, NCH₂CH₂CH₂CH₃ (TBA)); 2.13 (8H, m, NCH₂CH₂CH₂CH₃ (TBA)); 3.66 (8H, m, NCH₂CH₂CH₂CH₃ (TBA)); 3.85-5.21 (5H, m, alginate).

### EXAMPLE 3

### RAFT Functionalization of TBA-Alginate

1′-Carbonyldiimidazole (CDI) (1 g, 6 mmol) and BM1430 (2.17 g, 6 mmol) were dissolved in 10 mL of anhydrous DMSO, placed in a round bottom flask and left to react for 1 hour under nitrogen. Separately, TBA-alginate (1.3 g, 6 mmol hydroxyl groups) was dissolved in a 30 mL solution of 2% tetrabutylammonium fluoride in DMSO. The solution of CDI and BM1430 was then added dropwise to the TBA-alginate solution and the mixture was allowed to react at 40° C. for 24 hours. The product was then precipitated in cold 0.01M HCl in an ethanol/methanol (1:1) mixture, centrifuged, washed several times with cold ethanol/methanol (1:1), neutralized with sodium carbonate, then lyophilized from water. The experiment was performed for both the depolymerized and high molecular weight alginate. The alginate-BM140 products were dissolved in D₂O and analyzed by ¹H NMR to quantify the degree of substitution (DS) of hydroxyl groups to RAFT functionalities, which was found to be 0.03 for the high molecular weight alginate and 0.14 for the depolymerized alginate. ¹H NMR (400 MHz, D₂O, δ, ppm); 0.86 (3nₗH, t, CH₃(CH₂)₁₁S), 1.05-1.23 (23nₗH, m, CH₃(CH₂)₁₀CH₂S and SCH₂(CH₃)CO); 2.58 (2n₁H, t, CH₃(CH₂)₁₀CH₂S); 3.09-4.30 (5H, m, alginate); 4.98 (n₁H, s, S(CH₃)CHCO) where nₗ is the % functionalization of the RAFT moieties.

### EXAMPLE 4

Polymerization of PEGMA on alginate-BM1430 Separately, both variants of alginate-BM1430 (0.2 g), PEGMEMA (300 molar equivalent), and AIBN (0.1 molar equivalent) were dissolved in 50 mL of toluene and sealed in a round bottom flask. The mixtures were degassed at room temperature with nitrogen for 1 hour, then left to react at 70° C. for 3 hours. The polymerizations were terminated by cooling the solutions and exposing them to air, and the pure product was obtained by precipitating the solutions in cold diethyl ether/hexane (4:1) twice. After extensive drying, both polymers were characterized by GPC and static light scattering (dn/dc: 0.161) to determine the molecular weights.

### EXAMPLE 5

Alkyne functionalization of TBA-alginate TBA-alginate polymers generated in part (b) were combined with 4-oxo-4-(prop-2-ynyloxy)butanoic anhydride, triethyl amine (TEA), and 4-dimethylaminopyridine (DMAP) in a molar ratio of 1, 1 and 0.05 respectively to every hydroxyl function. The mixture was dissolved in a minimal amount of DMSO so as to just dissolve the solids, and left to react at 50° C. over 18 hours. The product was then precipitated in cold 0.01M HCl in an ethanol/methanol (1:1) mixture, centrifuged, washed several times with cold ethanol/methanol (1:1), neutralized with sodium carbonate, then lyophilized from water. The alginate-alkyne products were dissolved in D₂O and analyzed by ¹H NMR to quantify the degree of esterification of hydroxyl groups to alkyne functionalities and were found to be 0.32 alkyne moieties per alginate repeat unit for the high molecular weight alginate, and 0.24 alkyne moieties per repeat unit for the depolymerized alginate. FT-IR spectra were also recorded to verify the presence of alkyne moieties.

### EXAMPLE 6

Synthesis of poly(poly(ethylene glycol) methyl ether methacrylate) with terminal azides (PPEGMEMA-N₃) PEGMEMA (28.8 g, 96 mmol), was added to 20 mL of toluene, sealed in a round bottm flask, then degassed with nitrogen for 45 minutes. Separately, CuBr (0.138 g, 0.96 mmol) was degassed in a schlenk flask over three vacuum and nitrogen backfill cycles. PEGMEMA and toluene is then transferred to the schlenk flask with a cannula, then transferred to an oil bath at 70° C. PMDETA (0.334 g, 0.96 mmol), followed by ethyl 2-bromoisobutyrate (0.175 g, 0.96 mmol) were added with a gas tight syringe and the solution was reacted for 20 minutes. The polymerization was terminated by cooling the solution and exposing it to air. The product was passed over a short pad of alumina to remove copper and the pure bromide capped polymer was obtained by precipitating the solution in cold diethyl ether/hexane (4:1) twice. After extensive drying, the polymer was characterized by GPC and to determine the molecular weight and was found to be 25 kDa with a PDI of 1.31. Static light scattering (dn/dc: 0.161) was also performed and the reported molecular weight was 147 kDa. Subsequently, all of the polymer was dissolved in acetone, placed in a round bottomed flask with sodium azide (5 g, 76 mmol) and refluxed for 18 hours. The product was then reprecipitated twice in cold diethyl ether/hexane (4:1). An FT-IR spectrum was recorded to verify the presence of azide moieties.

### EXAMPLE 7

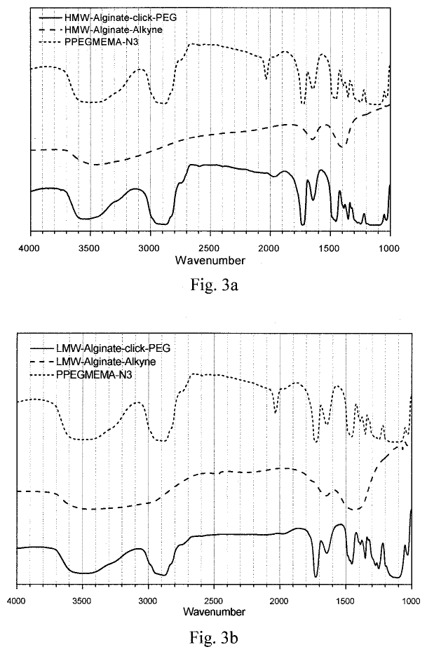

Copper catalyzed Azide-Alkyne conjugation of PPEGMEMA-N₃ to alginate-alkyne The alkyne functionalized alginate polymers from part (e) were combined with a molar equivalent of PPEGMEMA-N₃ to alkyne functions, equimolar ratio of CuSO₄ to alkyne functions, and 5 molar equivalent of sodium ascorbate to CuSO₄. This mixture was dissolved in DMSO with 10% of distilled water and placed in an oil bath at 45° C. for 3 days. The product was then dialysed against water with 4 water changes over 24 hours to remove CuSO₄ and sodium ascorbate and then freeze-dried. Thereafter, both polymers were characterized by GPC and static light scattering (dn/dc: 0.161) to determine the molecular weights, while FT-IR spectra were recorded to verify the disappearance of the azide peaks and the appearance of a triazole (FIG. 3a and b).

cl Example 8

Loading experiments of 4-n-butylresorcinol The optimum alginate-graft-PPEGMEMA to 4-n-butylresorcinol ratio for ideal encapsulation was determined via two different calcium mediated assembly methods. Method 1: 1 mL solutions of alginate-graft-PPEGMEMA at a concentration of 2 mg/mL were prepared with 4-n-butylresorcinol at mass ratios of 2, 1, 0.5, 0.25 and 0.125 and were allowed to mix for 24 hours. To each of the solutions, 0.111g of CaCl₂ was added and the mixtures were allowed to mix over 18 hours. The solutions were then centrifuged and the supernatants were analyzed for resorcinol concentration by UV/Vis spectrometry with a peak absorbance at 281 nm Method 2: 4-n-butylresorcinol encapsulation was performed by preparing 0.5 mL solutions of alginate-graft-PPEGMEMA at a concentration of 10 mg/mL with 4-n-butylresorcinol at mass ratios of 2, 1, 0.5, 0.25 and 0.125. Each sample (0.4 mL) was added to 1.6 mL of 1M CaCl₂ solutions under mixing, vortexed and then centrifuged. The supernatants were analyzed for resorcinol concentration by UV/Vis spectrometry.

### EXAMPLE 9

Release studies of 4-n-butylresorcinol loaded alginate-graft-PPEGMEMA Nano-assemblies were prepared for the alginate-graft-PPEGMEMA with 4-n-butylresorcinol at mass ratios of 1 and 0.125 by both techniques as highlighted in part (h). The supernatant was removed after centrifugation and washed once with methanol or 1M CaCl₂ for method 1 and 2 respectively, then centrifuged again. The solids were redispersed in a sodium acetate buffer at pH of 5 at a concentration of 5 mg/mL and then placed in a water bath at 37° C. A sample was removed at pre-determined intervals and centrifuged for 24 hours. The supernatant was analyzed for the concentration of 4-n-butylresorcinol by UV/Vis spectrometry. The experiments were performed in triplicate.

### EXAMPLE 10

### Results & Discussion

Sodium alginate is only soluble in aqueous solutions of a pH of 7 or higher thus there are only limited options in polymer functionalization. In addition to this, alginate, once dissolved in water results in a viscous solution which could pose processability issues. Therefore, alginate was depolymerized by reacting it with hydrogen peroxide and pyridine. While the molecular weight of alginate is 77.7 kDa, the molecular weight of our depolymerized alginate was 46.5 kDa.

In order to perform organic reactions on alginates such as esterifications, a solubility modification of sodium alginate to tetrabutylammonium alginate was performed (Scheme 1). Both the depolymerized lower molecular weight (LMW) and untreated higher molecular weight (HMW) sodium alginate were first acidified with hydrochloric acid, then reacted with tetrabutylammonium hydroxide (TBAOH) to yield the tetrabutylammonium (TBA) salt of alginate. Solubility studies in DMF, DMSO and THF was performed and the polymers showed solubility of at least 50 mg/mL was observed for DMF and DMSO with 2% tetrabutylammonium fluoride. The polymers were not soluble in THF.

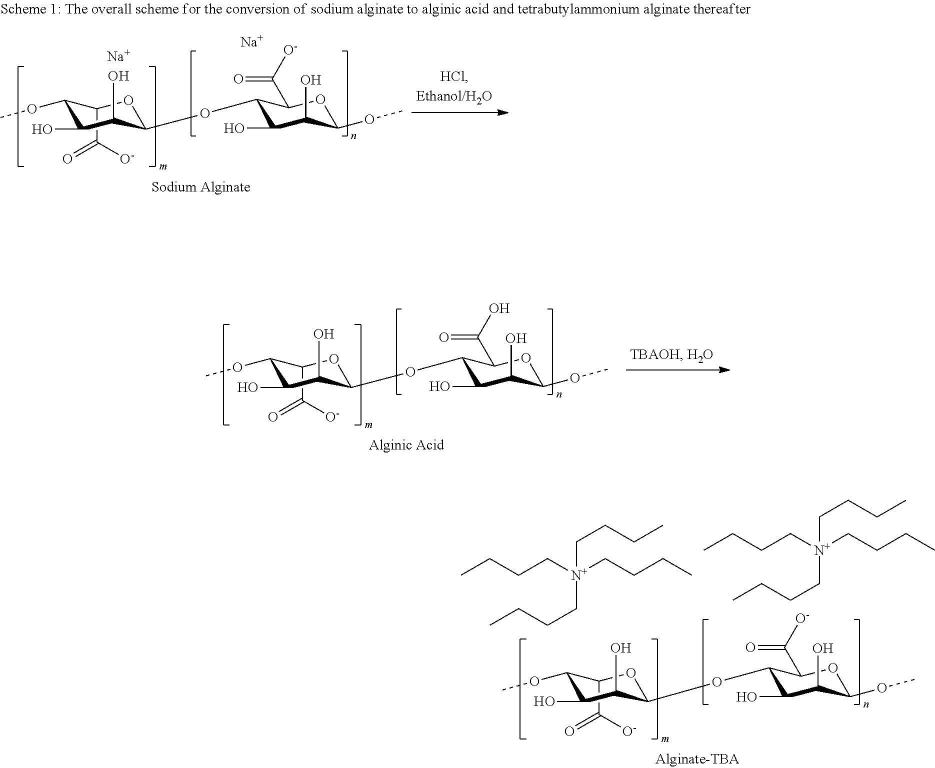

Both HMW and LMW TBA alginate were then reacted with BM1430, a commercially available RAFT acid, with CDI as an intermediate coupling agent. The resultant yellow coloured product was found to have 57% and 30% of RAFT moieties on the HMW and LMW alginate respectively, as determined by ¹H NMR by intergrating the protons from the RAFT moieties against the protons on each alginate repeat unit.

Polymer grafting was performed to the alginate based macroRAFT agents. PEGMEMA was polymerized in the presence of AIBN with DMSO as the solvent yielding comb block copolymers (Scheme 2). For HMW alginate-graft-PPEGMEMA, the Mₙ^{SLS} was 245 kDa, which corresponds to a DPₙ of 68 for the PPEGMEMA chains. For LMW alginate-graft-PPEGMEMA, the Mₙ^{SLS} was 252 kDa, which corresponds to a DPₙ of 67 for the PPEGMEMA chains.

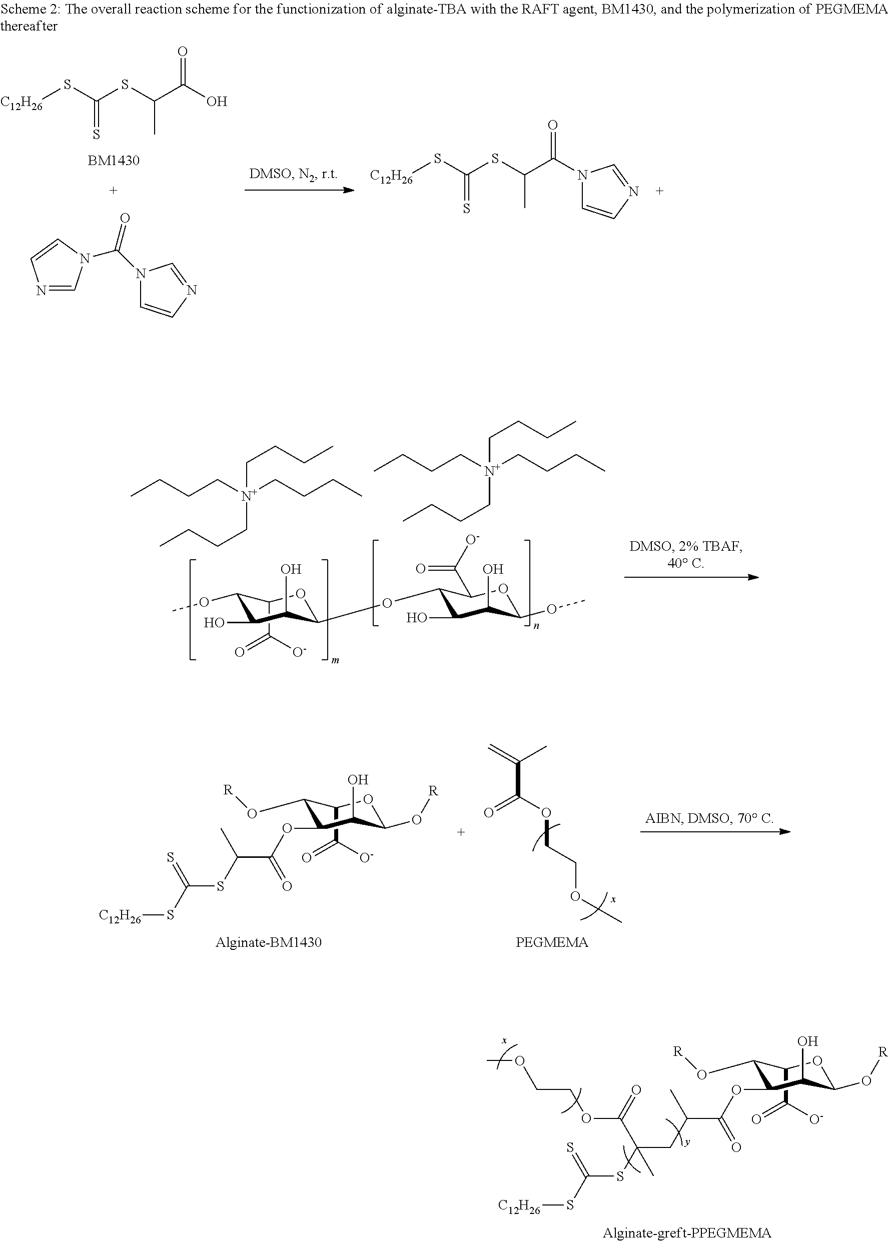

The macroRAFT approach to the synthesis of the comb alginate-graft-PPEGMEMA was shown to be successful. However, another method for polymer grafting was demonstrated via the ‘click’ chemistry approach. Firstly, alginate TBA was reacted with an alkyne anhydride to yield alkyne functionalized alginate (30% and 17% for HMW and LMW alginate respectively, as found by ¹H NMR). Separately, PPEGMEMA was synthesized by the atom transfer radical polymerization (ATRP) process, yielding bromide terminated polymer chains with a Mₙ^{GPC} of 25 kDa. The terminal bromides were converted into azides by refluxing the polymer with sodium azide overnight. These azide terminated chains were then reacted with the alkyne functionalized alginates in an equal stoichiometric ratio of azides and alkynes in the presence of CuSO₄ and sodium ascorbate as the reducing agent. The overall reaction is presented in Scheme 3. The product, analyzed by FT-IR, shows the complete disappearance of the R—N₃ stretch at 1970 nm in the final product, signifying a quantitative conversion of all azides and alkynes into triazoles (FIG. 3).

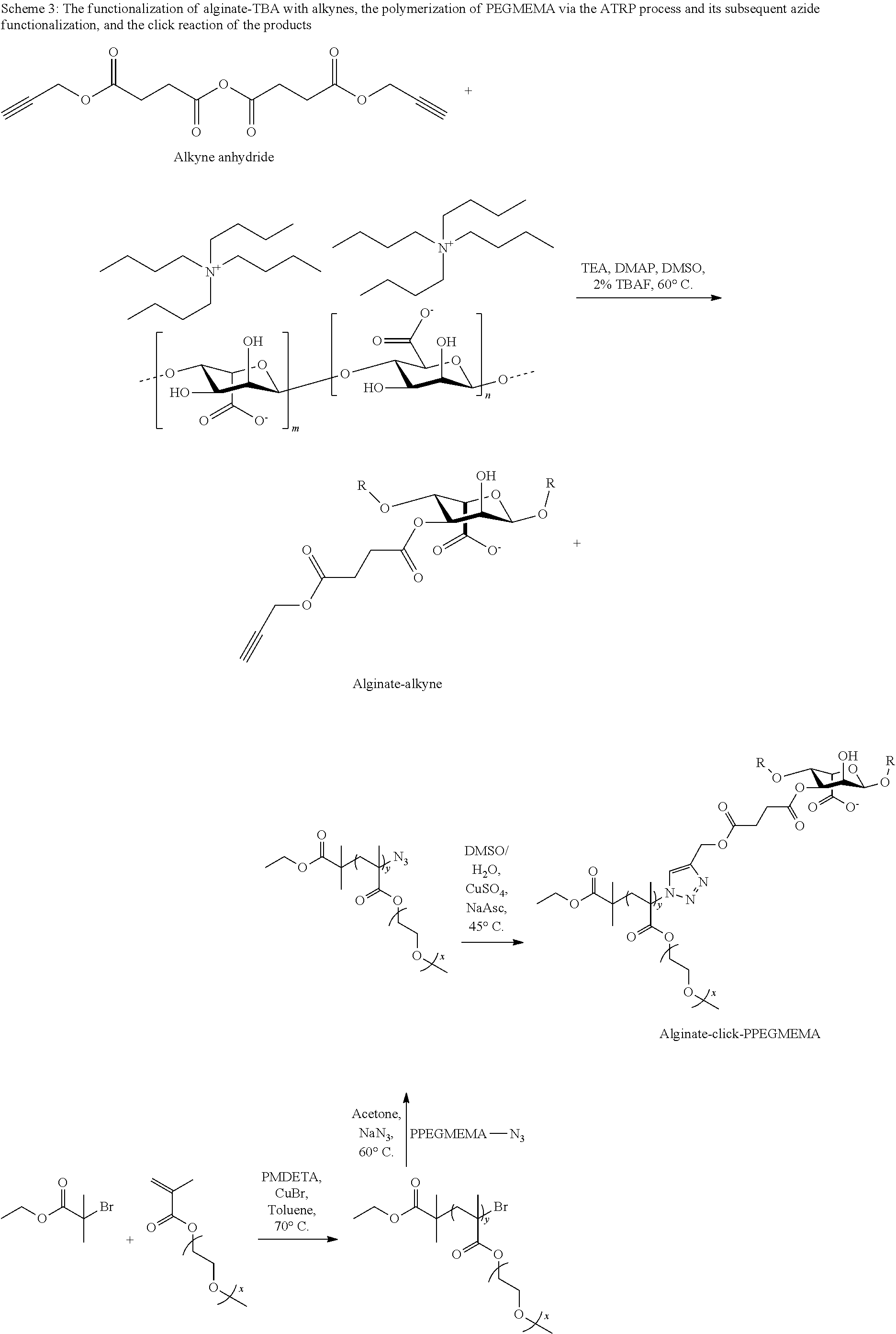

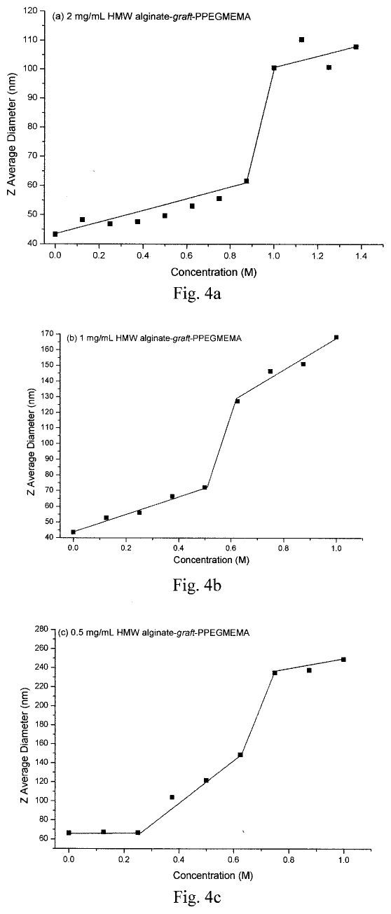

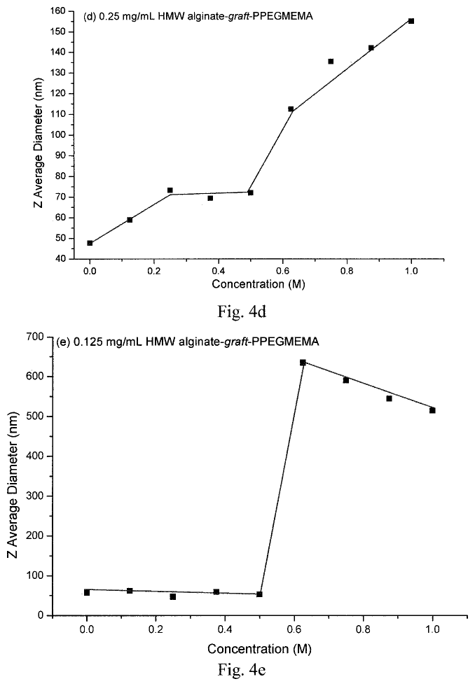

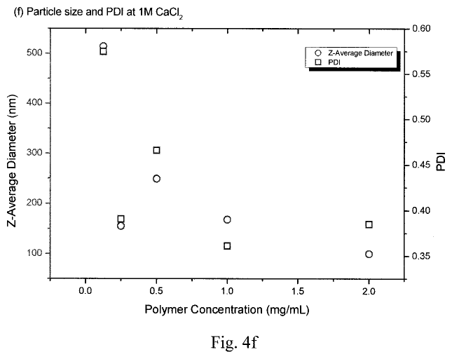

The polymers were then tested for their ability to self-assemble in the presence of calcium. Alginate-graft-PPEGMEMA samples were dissolved at a range of concentrations in water. Their hydrodynamic radius was measured by dynamic light scattering while calcium chloride was added to these solutions, raising its concentration at 0.125M increments. The particle size against calcium concentration for the HMW samples is highlighted in FIG. 4.

The general trend is that there is initially a gradual increase in particle size upon the addition of calcium up until a point where there is a sudden jump in particle size and thereafter a gradual increase once again. The particle size at 1M CaCl₂ reduces from 500 nm to 100 nm and the PDI decreases from 0.57 to 0.37 with an increase in polymer concentration.

The sudden increase in particle size suggests that there is a self-assembly or aggregation mechanism, presumably due to the stabilization of the PPEGMEMA chains. The point of sudden increase in particle size is regarded as the critical calcium concentration (CCC) required for polymer self-assembly and the average value for the analyzed polymers is 0.6M. The relationship of particle size and PDI to polymer concentration suggests that the larger concentration of polymer could provide a greater number of nucleation sites, thereby resulting in smaller particle sizes.

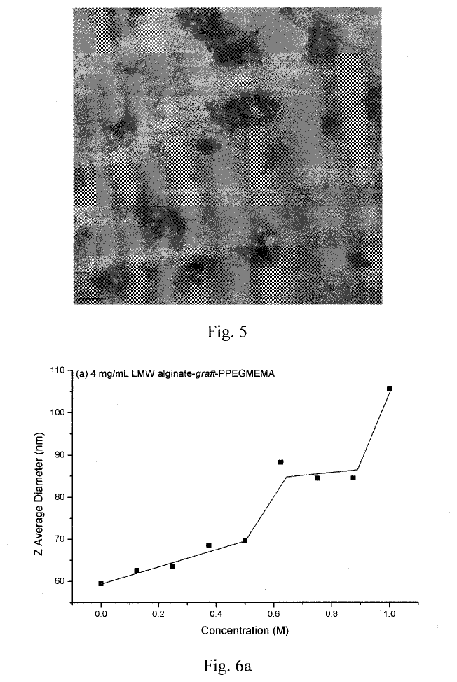

A TEM image was taken of the 1 mg/mL sample (FIG. 5) which shows 200 nm nanoassemblies. These nanoassemblies do not have any classical morphologies, however it shows that there are polymeric aggregates with a dense core, which likely corresponds to calcium cores.

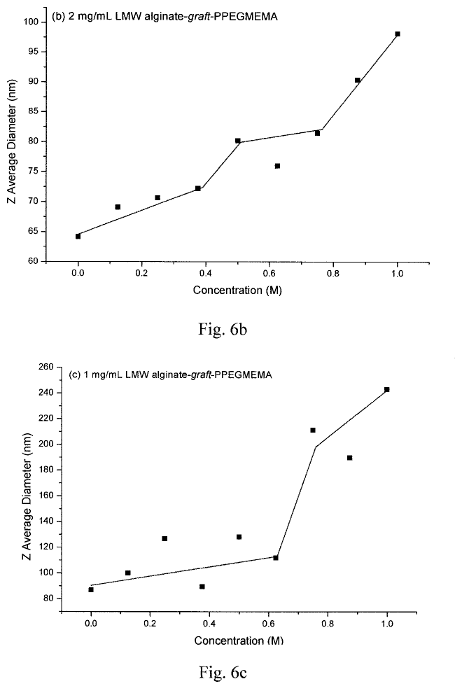

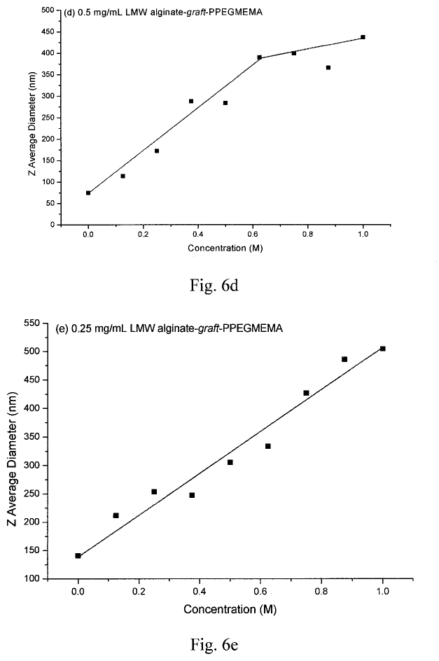

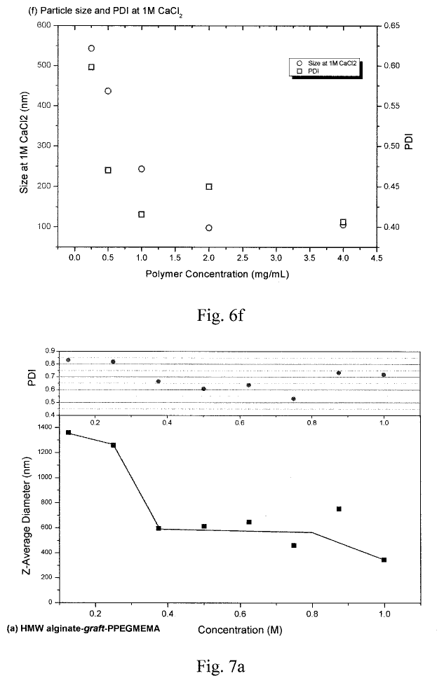

Similarly, the size relationship of calcium concentration to LMW alginate-graft-PPEGMA polymers was also measured and presented in FIG. 6. However, in the case of the LMW samples, the sudden jump in particle size is less pronounced as compared to the HMW samples, particularly at lower polymer concentrations. This could be attributed to the longer PPEGMEMA chains and their influence on the self-assembly of the comb copolymer. It is likely that the longer chains could be causing aggregation of the polymers without a critical point to cause a directed self-assembly. However, FIG. 6e shows that the particle size and PDI both decrease as the polymer concentration increases, in agreement with the data for the HMW samples suggesting the same nucleation dominant self-assembly mechanism.

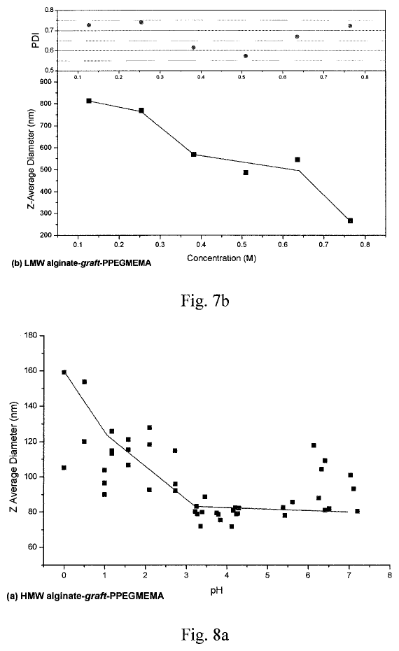

An alternative strategy towards directing these polymer nanoassemblies would be to add the polymers to a calcium solution. A 2M solution of CaCl₂ was prepared, to which a 10 mg/mL polymer solution was added. The particle size was measured and reported in FIG. 7. There is an agreement that the previous results highlighted in FIGS. 4 and 6 that the concentration of polymer had an inverse effect to the particle size and the PDI. However, the PDIs are much broader than previous experiments and could be due to the fact that the nucleation and cross-linking of alginate is an irreversible process, and hence the kinetics would differ significantly for this titration.

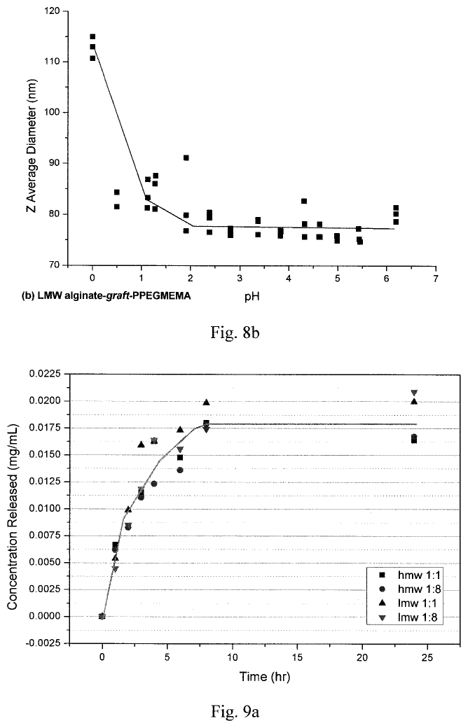

It was also hypothesized that the carboxylic acid functionality on the alginate backbone could illicit a pH response. Therefore, 2 mg/mL solutions of both HMW and LMW alginate-graft-PPEGMEMA were titrated against concentrated HCl from its base pH of 6.5-7 down to 0. The HMW sample (FIG. 8a) showed a general increase in particle size with decreasing pH, however the curve fit is poor, which could suggest numerous mechanisms at play. The LMW sample (FIG. 8b) however showed a sudden jump in particle size below a pH of 1, alluding to the likelihood that the alginate-graft-PPEGMEMA could self-assemble at a low pH.

Finally, encapsulation studies were performed on the model compound, 4-n-butylresorcinol (4BR), which is a cosmetic bleach with poor water solubility and high skin irritability. A controlled release of 4BR would be advantagous in cosmetic applications in order to achieve high dosing concentrations while mitigating skin irritation. The alginate-graft-PPEGMEMA was dissolved in methanol along with a range of ratios of 4BR. The encapsulation of 4BR in alginate was performed by either introducing calcium into the methanol solution of 4BR and alginate, or by introducing the methanol solution of 4BR and alginate into 2M CaCl₂.

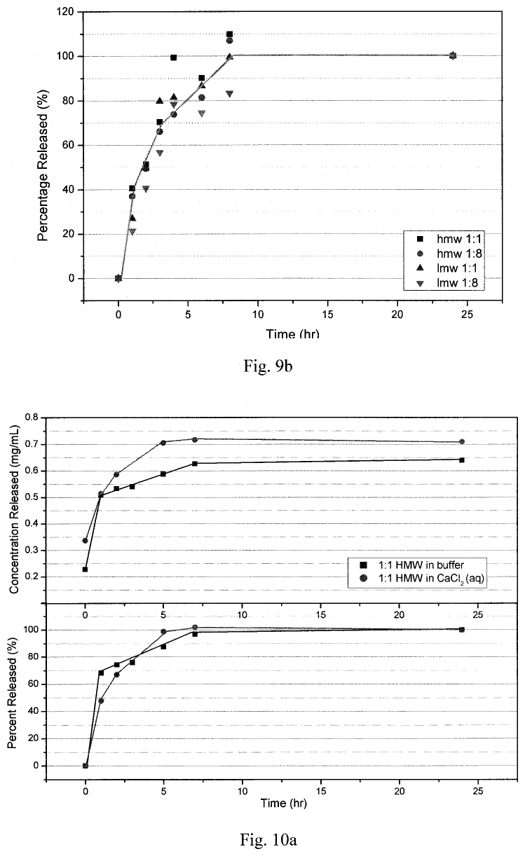

Table 1 illustrates the encapsulation efficiency of the alginate polymers when calcium is introduced to the methanol solution. The percentage of encapsulation is the highest for the 1:8 ratio of polymer to 4BR, and the encapsulation efficiency remains fairly constant otherwise. The results suggest a highly efficient encapsulation process. From this data, the nanoassemblies with a 1:1 and 1:8 ratio were introduced to solutions of sodium acetate buffer (pH=5) equilibrated at 37° C. Samples were taken at pre-determined intervals, centrifuged, and the supernatant was analyzed by UV/Vis spectroscopy for the concentration of 4BR (λ=280 nm). The release of 4BR is reported in FIG. 9. A plateau in released concentration of 4BR is observed after 6 hours, and the total concentration plateaus at the same amount for all polymer to resorcinol concentrations, which further suggests that the simple encapsulation data reported in Table 1 also factors in 4BR that could be precipitating upon the addition of CaCl₂.

TABLE 1
Encapsulation percentages for the various ratios
of 4BR to HMW alginate-graft-PPEGMEMA when CaCl₂
is introduced into the polymer solution

| | Ratio of | Encap | Loading | % |
|---|---|---|---|---|
| | 4BR:poly | (%) | (%) | wasted |
| HMW | 1:08 | 87 | 11 | 13 |
| | 1:04 | 68 | 17 | 32 |
| | 1:02 | 67 | 33 | 33 |
| | 1:01 | 64 | 64 | 36 |
| | 2:01 | 68 | 136 | 32 |

Table 2 illustrates the encapsulation efficiency of the alginate polymers when the polymer and 4BR containing methanol solution is introduced to a calcium solution. The percentage of encapsulation is the highest for the 1:2 ratio of polymer to 4BR, and the encapsulation efficiency remains fairly constant otherwise.

TABLE 2
Encapsulation percentages for the various ratios of 4BR to HMW
alginate-graft-PPEGMEMA and LMW alginate-graft-PPEGMEMA
when the polymer solution in methanol is introduced to 2M CaCl₂

| | Ratio of | Encap | Loading | % |
|---|---|---|---|---|
| | poly:4BR | (%) | (%) | wasted |
| HMW | 1:08 | 83 | 10 | 17 |
| | 1:04 | 73 | 18 | 27 |
| | 1:02 | 85 | 42 | 15 |
| | 1:01 | 80 | 80 | 20 |
| | 2:01 | 76 | 152 | 24 |
| LMW | 1:08 | 72 | 9 | 28 |
| | 1:04 | 69 | 17 | 31 |
| | 1:02 | 83 | 42 | 17 |
| | 1:01 | 78 | 78 | 22 |
| | 2:01 | 77 | 154 | 23 |

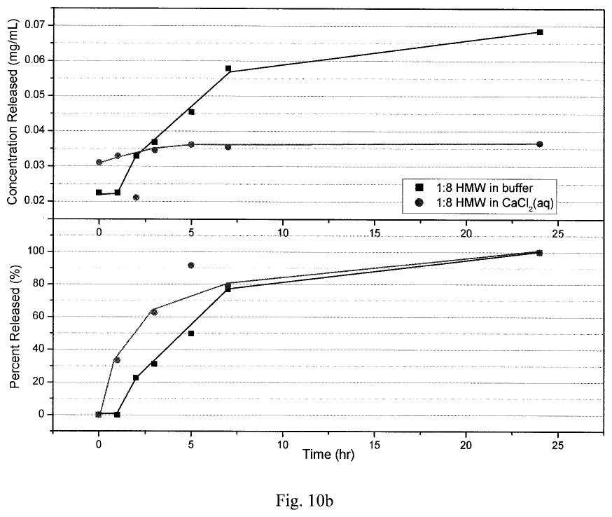

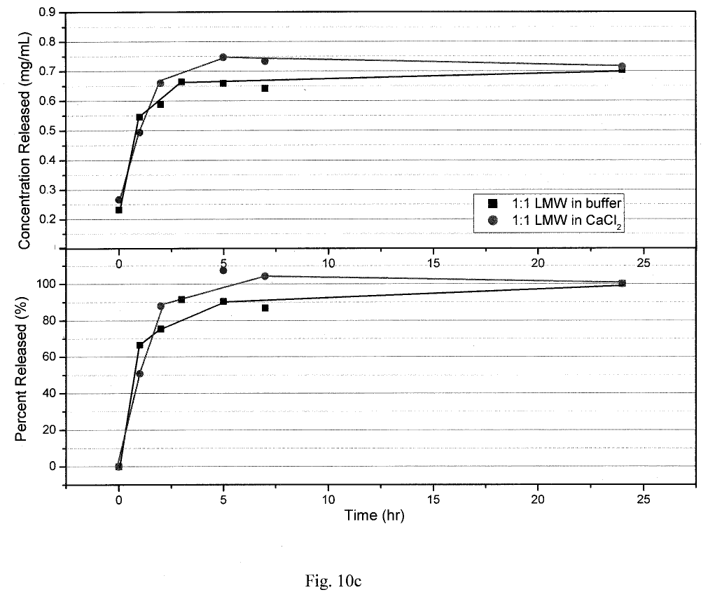

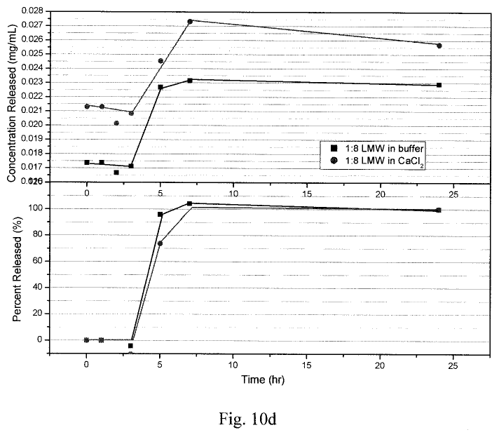

For the sake of comparison, the nanoassemblies with a 1:1 and 1:8 ratio were introduced to solutions of either sodium acetate buffer (pH=5) equilibrated at 37° C. or 2M CaCl₂ at room temperature as a control. Samples were taken at pre-determined intervals, centrifuged, and the supernatant was analyzed by UV/Vis spectroscopy for the concentration of 4BR (λ=280 nm). The release of 4BR is reported in FIG. 10. The release mechanism shows similar release kinetics for the 1:1 samples in both the buffer and 2M CaCl₂. However, it can be observed that there is a higher initial burst of 4BR release for the samples in buffer for all samples. Also, a significant release of 4BR for the 1:8 HMW sample can only be observed in buffer when compared to the sample in CaCl₂.

Alternatively, the polymerization may be undertaken with OEGMA, resulting in a POEGMA copolymer of the modified alginate, as laid out in the following.

### EXAMPLE 11

### Polymerization of OEGMA on Alginate-BM1430

Both variants of alginate-BM1430 (0.2 g), OEGMEMA (300 molar equivalent), and AIBN (0.1 molar equivalent) were dissolved in 50 mL of toluene and sealed in a round bottom flask. The mixtures were degassed at room temperature with nitrogen for 1 hour, then left to react at 70° C. for 3 hours. The polymerizations were terminated by cooling the solutions and exposing them to air, and the pure product was obtained by precipitating the solutions in cold diethyl ether/hexane (4:1) twice. Thereafter they were redissolved in water and dialyzed for two days, then freeze dried. These polymers were further analysed by triple detection THF GPC and static light scattering (SLS) to gather a deeper understanding of polymer size due to the complexity of the macromolecule. The average do/dc value in THF at 40° C. of the copolymers was found to be 0.059 and molecular weight determination by GPC and SLS showed that the molecular weight of the HMW polymer was Mn^{T-GPC}:659 kDa (Ð: 2.37), Mₙ^{SLS}:547 kDa, and that of the LMW polymer was Mₙ^{T-GPC}:870 kDa (Ð: 2.17), Mₙ^{SLS}:753 kDa. ¹H NMR (400 MHz, D20, δ, ppm); 0.63-1.10 (3n₂H, CH₃CCH₂ backbone); 1.46 (16H, CH₃(CH₂)₈C₃H₆S); 1.61-2.09 (2n₂H, CH₃CCH₂ backbone); 3.28 (3n₂H+3n₃H, CH₃OCH₂); 3.39-3.86 (34n₂H+34n₃H, COOCH₂CH₂O(CH₂—CH₂O)₈CH₃); 3.91-4.27 (2n₂H+2n₃H, COOCH₂CH₂O(CH₂—CH₂O)₈CH₃); 5.48 (n₃H, HHC═C(CH₃) monomer) 6.03 (n₃H, HHC═C(CH₃) monomer) where n₂ is the DPn of the polymer and n₃ is the number of moles of unreacted monomer per RAFT moiety.

### EXAMPLE 12

### Loading Experiments of 4-n-butylresorcinol

The optimum alginate-graft-POEGMEMA to 4-n-butylresorcinol ratio for ideal encapsulation was determined via two different calcium mediated assembly methods. Method 1: 1 mL solutions of alginate-graft-POEGMEMA at a concentration of 2 mg/mL⁻¹ in methanol were prepared with 4-n-butylresorcinol at mass ratios of 2, 1, 0.5, 0.25 and 0.125 (i.e. 4BR concentration of 4, 2, 1, 0.5 and 0.25 mg/mL⁻¹, respectively) and were allowed to mix for 24 hours. To each of the solutions, 0.111g of CaCl₂ (1 M in solution) was added and the mixtures were allowed to mix over 18 hours. The solutions were then centrifuged and the supernatants were analyzed for resorcinol concentration by UV/Vis spectrometry with a peak absorbance at 281 nm while the pellet was isolated for release studies outlined in Example 13. Method 2: 4-n-butylresorcinol encapsulation was performed by preparing 0.5 mL solutions of alginate-graft-POEGMEMA at a concentration of 10 mg/mL⁻¹ in methanol with 4-n-butylresorcinol at mass ratios of 2, 1, 0.5, 0.25 and 0.125 (i.e. 4BR concentration of 4, 2, 1, 0.5 and 0.25 mg/mL⁻¹, respectively). Each sample (0.4 mL) was added to 1.6 mL of 1 M CaCl₂ solutions under mixing, vortexed and then centrifuged. The supernatants were analyzed for resorcinol concentration by UV/Vis spectrometry with a peak absorbance at 281 nm while the pellet was isolated for release studies outlined in Example 9.

### EXAMPLE 13

### Release Studies of 4-n-butylresorcinol Loaded Alginate-Graft-POEGMA

The isolated nanoparticles obtained by both methods in Example 8 with alginate-graft-POEGMA to 4-n-butylresorcinol mass ratios of 1 and 0.125 were washed once with methanol or 1 M CaCl₂ for method 1 and 2 respectively, then centrifuged again. The solids were redispersed in a sodium acetate buffer at a pH of 5 at a concentration of 5 mg mL⁻¹ and then placed in a water bath at 37° C. A sample was removed at pre-determined intervals and centrifuged for 24 hours. The supernatant was analysed for the concentration of 4-n-butylresorcinol by UV/Vis spectrometry. The experiments were performed in triplicate.

Sodium alginate is an interesting polymer due to its non-toxicity, biodegradability and derivatization from renewable sources. However, it is only soluble in aqueous solutions of a pH of 7 and does not dissolve readily. It affords a viscous aqueous solution, which poses challenges in polymer processability and functionalization, thus there are only limited options in polymer functionalization. The high viscosity and poor solubility of alginate solutions are due to its high molecular weight, which is 279 kDa and 213 kDa (Ð: 3.31) as determined by SLS and GFC, respectively, and strong intra and inter chain hydrogen bonding. Kapishon et al. recognized this effect and in their work, only focused on functionalizing depolymerized alginate. In the depolymerization process, they showed that the degradation of polymer size is time dependent and after 90 minutes, they obtained alginate of a molecular weight of 28 kDa. In this disclosure, a polymer was obtained with a molecular weight of 96 kDa and 73 kDa (Ð: 2.43) by SLS and GFC, respectively, under the same conditions. In this disclosure, studies were conducted on both the pristine as well as the depolymerized alginate and will be discussed in this section.

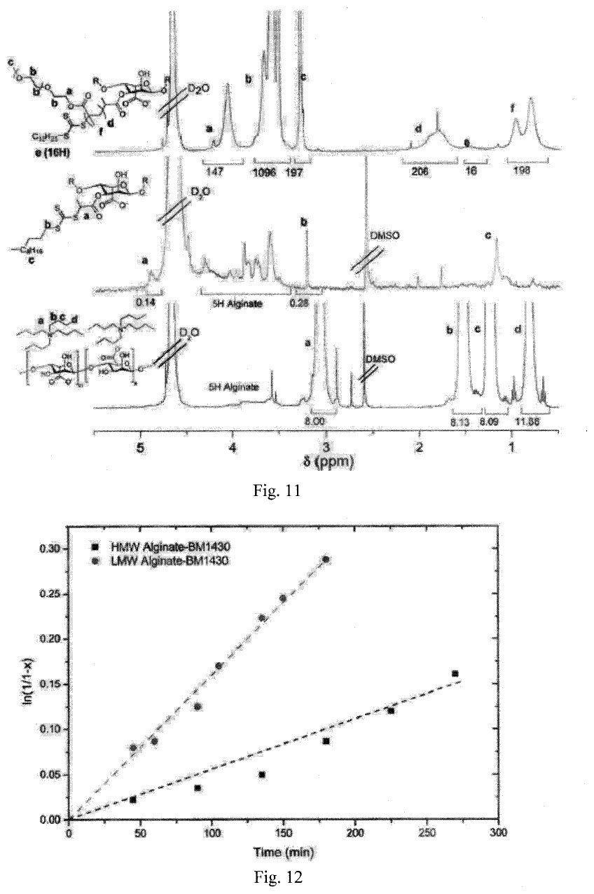

To afford solubility of alginate in organic solvents, a counter-ion modification of sodium alginate to tetrabutylammonium alginate was performed (Scheme 1). Both the depolymerized lower molecular weight (LMW) and untreated higher molecular weight (HMW) sodium alginate were first acidified with hydrochloric acid, and then reacted with tetrabutylammonium hydroxide (TBAOH) to yield the tetrabutylammonium (TBA) salt of alginate. Solubility studies in DMF and DMSO were performed and the polymers showed solubilities of at least 50 mg mL⁻¹ in DMF and DMSO with 2% tetrabutylammonium fluoride. HMW and LMW TBA alginate was then functionalized with BM1430, a commercially available RAFT acid, with CDI as an intermediate coupling agent (Scheme 2). The resulting yellow coloured product had a degree of substitution (DS) of 0.03 and 0.14 on the HMW and LMW-alginate, respectively, as determined by ¹H NMR by integrating the protons from the RAFT moieties against the protons on each alginate repeat unit (FIG. 11). The LMW sample had a higher DS than the HMW sample, which suggests that the inherently higher viscosity due to the larger molecular weight acts as a hindrance towards the functionalization of the alginate backbone.

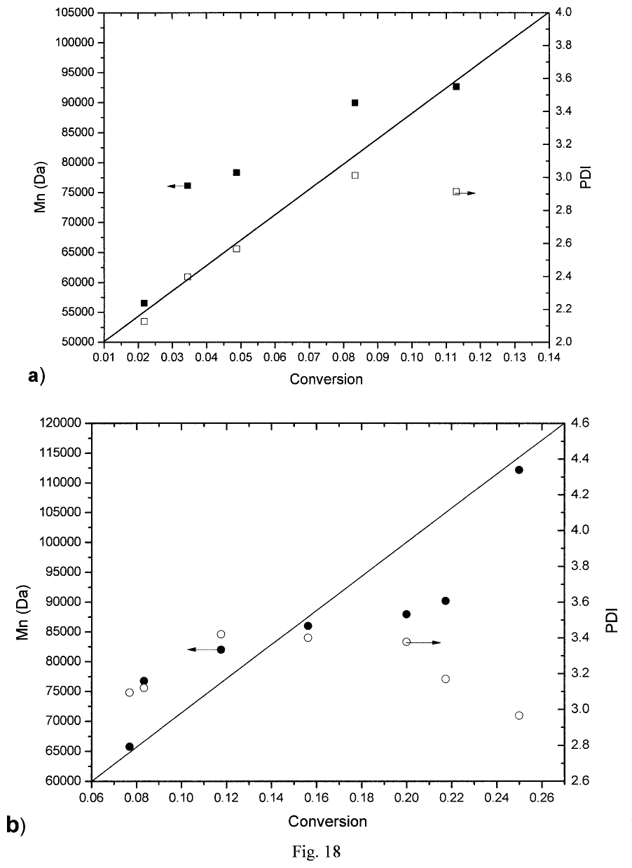

OEGMA was grafted to the alginate macroRAFT agents at 65° C. in the presence of AIBN with toluene as the solvent yielding comb block copolymers (FIG. 2b). Small aliquots were removed at intervals of 45 minutes from each of these reactions, exposed to air and cooled. Solvent was removed from the samples under vacuum, and they were analysed by GPC in THF and ¹H NMR in CDCl₃ to determine their molecular weights and conversion, respectively. The data are summarized in Table 3 while FIG. 12 and FIG. 18 show the first-order kinetic plot and molecular weight development with conversion, respectively.

TABLE 3
Summary of reaction time, conversion and degree of polymerization
(DPn) determined by ¹H NMR, Mₙ^{GPC} and its associated PDI (Ð) as
determined by size exclusion chromatography with a single refractive
index detector and PMMA calibration

| | | Reaction | Conversion | | Mₙ^{theo} | Mₙ^{GPC} | |
|---|---|---|---|---|---|---|---|
| | No. | Time | (%) | DPn | (kDa) | (kDa) | Ð |
| HMW | 1 | 45 | 2.17 | 5 | 267 | 56.5 | 2.13 |
| | 2 | 90 | 3.45 | 11 | 333 | 76.1 | 2.40 |
| | 3 | 135 | 4.88 | 16 | 387 | 78.3 | 2.57 |
| | 4 | 180 | 8.33 | 25 | 485 | 89.9 | 3.01 |
| | 5 | 225 | 11.29 | 35 | 594 | 92.6 | 2.91 |
| | 6 | 270 | 14.86 | 48 | 735 | 96.8 | 3.06 |
| LMW | 1 | 45 | 7.69 | 25 | 508 | 65.8 | 3.09 |
| | 2 | 60 | 8.33 | 35 | 682 | 76.7 | 3.12 |
| | 3 | 90 | 11.76 | 42 | 804 | 82.0 | 3.42 |
| | 4 | 105 | 15.63 | 50 | 943 | 86.2 | 3.40 |
| | 5 | 135 | 20.00 | 58 | 1,083 | 88.0 | 3.38 |
| | 6 | 150 | 21.74 | 62 | 1,152 | 90.2 | 3.17 |
| | 7 | 180 | 25.00 | 66 | 1,222 | 112.2 | 2.97 |

For both polymers, pseudo first-order kinetics and agreement between molecular weight evolution with conversion were observed, which suggests that the macroRAFT agents afforded good control of polymerization. The rate of polymerization on the HMW macroRAFT agent, observed from the gradient of the line of best fit, was much slower than that of the LMW macroRAFT agent. Once again, this is likely to be a result of the higher viscosity of the HMW sample, due to its larger molecular weight, which, in this case slows down the rate of polymerization. It is due to this that the polymerization on the HMW macroRAFT agent was allowed to proceed for a longer duration.

HMW polymer #6, with a reaction time of 270 minutes, and LMW polymer #4, with a reaction time of 105 minutes were used for further experiments due to their similar degree of polymerization (DPn). These polymers were precipitated in a mixture of hexane/diethyl ether (4:1), then dialyzed against water for 2 days, and finally lyophilized to remove all unreacted solvent and monomer. The final polymers were analysed by ¹H NMR (FIG. 11) as well as by SLS in water using a dn/dc value of 0.161. For HMW-alginate-graft-POEGMA, Mₙ^{GPC} was 96.8 kDa (Ð: 3.06), Mₙ^{SLS} was 547 kDa, DPn was 48, and Mₙ^{calc}, calculated from the sum of Mₙ^{GFC} and the size of each chain based on the DPn, was 735 kDa. Similarly, for LMW-alginate-graft-POEGMA, Mₙ^{GPC} was 86.2 kDa (Ð: 3.40), Mₙ^{SLS} was 753 kDa, DPn was 50, and Mₙ^{calc} was 943 kDa. It should be noted that the theoretical Mₙ of the polymer, calculated from the average DPn, is based on the assumption of 100% RAFT initiation efficiency, which might not necessarily be the case, and so the chains may actually be longer than what is calculated.44 The Mₙ^{GPC} values are around 6-10 times lower than Mₙ^{calc} and Mₙ^{SLS} for both polymers. This could be attributed to the complexity of polymer topology. To obtain the true molecular weight distribution of the polymers, triple detection GPC was used. Firstly, the dn/dc values of the polymers in THF was determined and found to be 0.059. From this dn/dc value, the molecular weight by triple detection was found to be 659 kDa (Ð: 2.37) and 870 kDa (Ð: 2.17) for the HMW and LMW-alginate-graft-POEGMA copolymers, respectively. All the molecular weight data are summarized in Table 4.

TABLE 4
A summary of all the different Mₙ values calculated by static
light scattering (Mₙ^{SLS}), gel filtration chromatography (Mₙ^{GFC}),
gel permeation chromatography (Mₙ^{GPC}), triple detection gel
permeation chromatography (Mₙ^{T-GPC}) and ¹H NMR(Mₙ^{calc})

| | Mₙ^{SLS} | Mₙ^{GFC} | Mₙ^{GPC} | Mₙ^{T-GPC} | Mₙ^{calc} |
|---|---|---|---|---|---|
| | (kDa) | (kDa) | (kDa) | (kDa) | (kDa) |
| HMW-alginate | 279 | 213 | — | — | — |
| LMW-alginate | 96 | 73 | — | — | — |
| HMW-alginate- | 547 | — | 96.8 | 659 | 735 |
| graft-POEGMA | | | | | |
| LMW-alginate- | 753 | — | 112.2 | 870 | 943 |
| graft-POEGMA | | | | | |

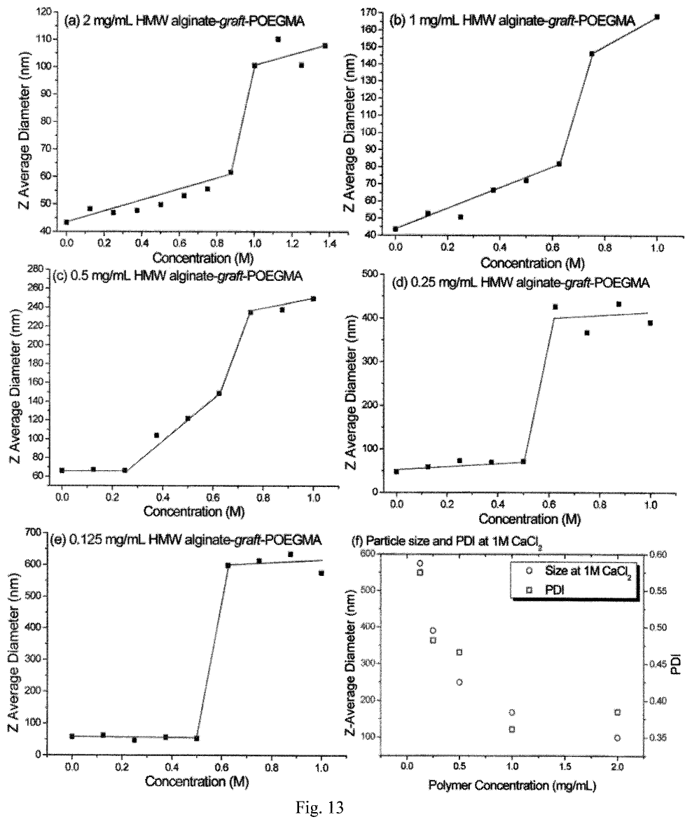

The polymers were then tested for their ability to self-assemble in the presence of calcium. Alginate-graft-POEGMA samples were dissolved at a range of concentrations in water. These solutions were titrated against CaCl₂ at 0.125 M increments and the hydrodynamic radius was measured by DLS. The particle size against calcium concentration for the HMW samples is highlighted in FIG. 13.

A gradual increase in particle size was observed upon the addition of calcium initially then a sudden jump in particle size and finally a gradual increase once again. With an increase in polymer concentration, the calcium concentration required for this jump increased, the particle size at 1 M CaCl₂ reduced from 500 nm to 100 nm and the PDI decreased from 0.57 to 0.37.

The jump in particle size suggests that there is a self-assembly or aggregation mechanism, similar to effects seen with LCST mechanisms, which would presumably be due to calcium mediated cross-linking. Since it is well established that the addition of calcium to alginate results in uncontrolled cross-linking gelation, the presence of the grafted POEGMA chains appears to be critical in restricting the increase of particle size to under 500 nm where they form the solubilizing moieties of the cross-linked alginate nanoparticle. The point of sudden increase in particle size, or the critical calcium concentration (CCC), required for polymer self-assembly increases with an increase in polymer concentration due to the increase in the number of binding sites for calcium. Furthermore, the relationship of particle size and PDI to polymer concentration is in agreement with work by Blandino et al., who show that the gelation kinetics of calcium alginate are dominated by the rate of diffusion of Ca²⁺ to the binding sites on alginate. With an increase in alginate concentration, the calcium would have more binding sites available per unit volume, which would yield a more densely cross-linked network, thereby resulting in smaller nanoassemblies, as is the general case for intramolecularly cross-linked nanoparticles.

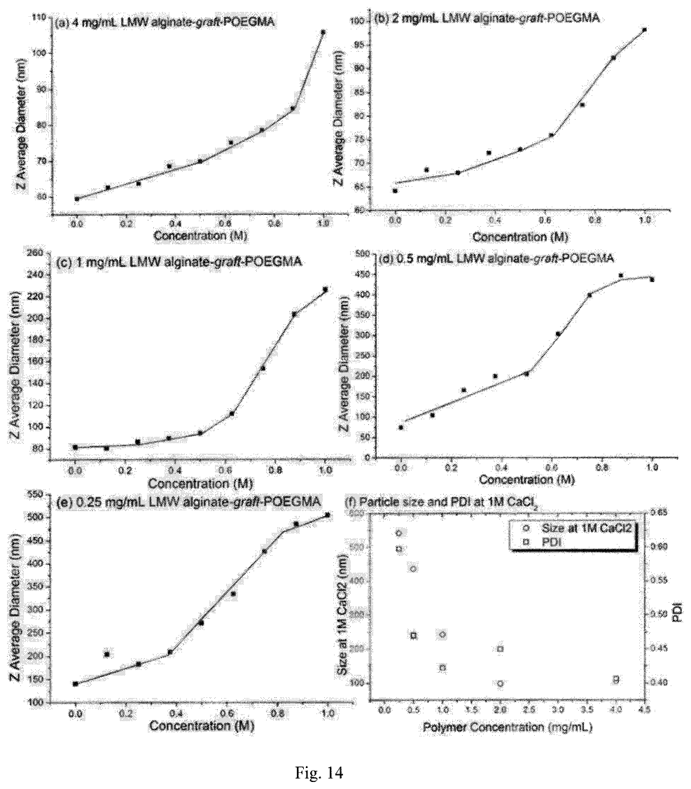

The size relationship of calcium concentration to LMW-alginate-graft-POEGMA polymers was also measured and is presented in FIG. 14. The data show a marginal increase in size initially, then a marked increase at a critical point with a smooth upward trend. This size relationship, and thereby assembly characteristics, differs for the HMW samples and can be attributed to the difference in alginate backbone length. However, the CCC required for this sharp increase in size was also directly proportional to the concentration of polymer in solution, with an inverse relationship between polymer concentration and final particle size and PDI (FIG. 14f). This similar trend to the HMW samples suggests the same nucleation dominant self-assembly mechanism.

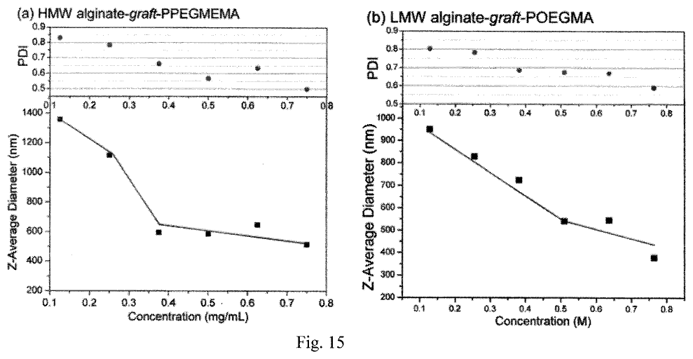

There was also investigated the evolution of particle size when polymer was added to calcium instead. A 1 M solution of CaCl₂ was prepared, to which a 10 mg mL⁻¹ polymer solution was titrated. The particle size, measured and reported in FIG. 15, decreased with increasing polymer, and began to stabilize at polymer concentrations of >0.4 mg mL⁻¹ The PDI of the particles also generally decreased, suggesting a dynamic normalization of particle size. The result is in agreement with previous results shown in FIGS. 13 and 14 where the concentration of polymer had an inverse effect on the particle size and the PDI. However, the PDIs are much broader than previous experiments, which could be due to the fact that the nucleation and cross-linking of alginate is an irreversible process.

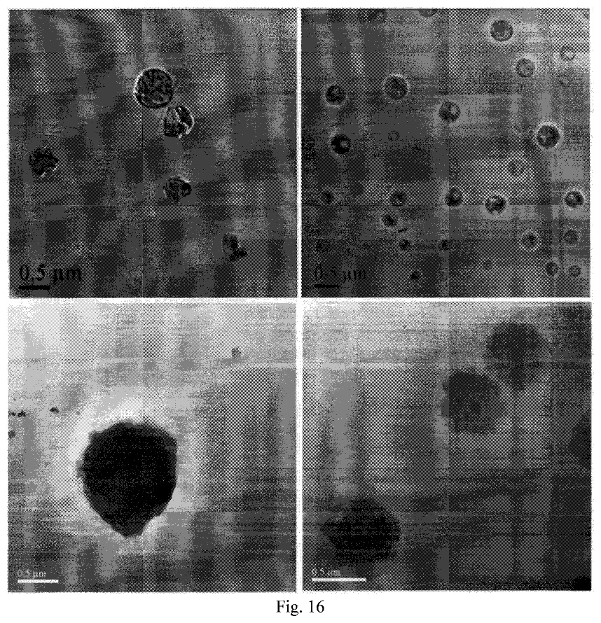

TEM images (FIG. 16) were taken of the samples obtained at the end of the aforementioned polymer addition to calcium self-assembly studies. The HMW alginate-graft-POEGMA particles measured an average of 500±100 nm and the LMW-alginate-graft-POEGMA particles an average of 375±75 nm The particle sizes are in agreement with the sizes found by DLS. Only the LMW particles are of classical spherical morphology, while the HMW particles have a varied morphology and are denser in appearance. This suggests a more ordered assembly for the LMW particles as opposed to a less ordered aggregation for the HMW particles. The morphology observed in these images further reiterates that the backbone length has a direct relationship with the self-assembly.

The carboxylic acid functionality on the alginate backbone could also elicit a pH triggered self-assembly due to the insolubility of alginic acid in water, and so, 2 mg mL⁻¹ solutions of both HMW and LMW-alginate-graft-POEGMA were titrated from their base pH of 6.5-7 down to 0. As shown in FIG. 8 a and b, both the samples show relatively different response curves, with a better fit to the predicted size evolution (line). Once again, it seems likely that the size of the backbone has a direct effect on whether these particles aggregate or assemble; however, this increase in particle size at such low pH could be dependent on factors other than self-assembly.

The synthesized polymers showed excellent solubility in a range of solvents, with DLS data showing a similar particle size across all of them (Table 5).

TABLE 5
Z-average particle size and polydispersity index
(PDI) of the 2 polymers in different solvents

| | | Z-average size | |
|---|---|---|---|
| | Solvent | (nm) | PDI |
| HMW Alginate-graft- | Water | 43.7 | 0.347 |
| POEGMA | Chloroform | 64.9 | 0.110 |
| | Methanol | 37.8 | 0.251 |
| | Acetone | 44.4 | 0.373 |
| LMW Alginate-graft- | Water | 43.6 | 0.381 |
| POEGMA | Chloroform | 37.9 | 0.189 |
| | Methanol | 45.95 | 0.256 |
| | Acetone | 31.5 | 0.186 |

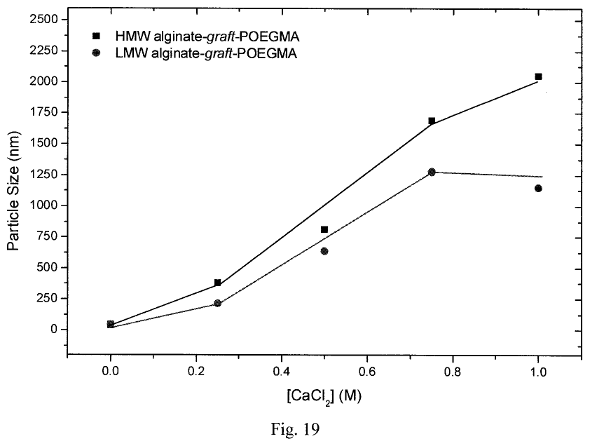

Polymers in methanol also showed the same relationship of particle size to calcium concentration as observed for the aqueous systems (FIG. 19). This performance shows tremendous potential for the encapsulation of lipophilic active compounds. An example is 4BR, which is a lipophilic skin therapeutic that causes skin irritation. A sustained release capsule would be ideal to minimize its negative effects and maximize solubility. Since 4BR is highly soluble in methanol, the disclosed polymers would be the ideal carrier to maximize its encapsulation.

To study the encapsulation of 4BR, alginate-graft-POEGMA polymers and 4BR were dissolved in methanol in different ratios. The encapsulation of 4BR in alginate was performed by either adding CaCl₂ to the methanol solution with a final concentration of 1 M, or introducing the methanol solution into 1 M CaCl₂. For the latter, a concentrated solution of the polymer and 4BR in methanol was prepared for the range of polymer to 4BR ratios, before introduction to CaCl₂ solutions. The samples were centrifuged to separate the solids, and the supernatant was analysed for 4BR concentration. Table 6 lists the 4BR to polymer ratios as well as the encapsulation efficiency and percent loading.

TABLE 6
Encapsulation efficiency and loading % for the various
ratios of 4BR to the HMW and LMW copolymer when the methanolic
solution is introduced to 1M CaCl₂

| | Ration of | Encap | Loading |
|---|---|---|---|
| | poly:4BR | (%) | (%) |
| HMW | 1:8 | 83 | 10 |
| | 1:4 | 73 | 18 |
| | 1:2 | 85 | 42 |
| | 1:1 | 80 | 80 |
| | 2:1 | 76 | 152 |
| LMW | 1:8 | 72 | 9 |
| | 1:4 | 69 | 17 |
| | 1:2 | 83 | 42 |
| | 1:1 | 78 | 78 |
| | 2:1 | 77 | 154 |

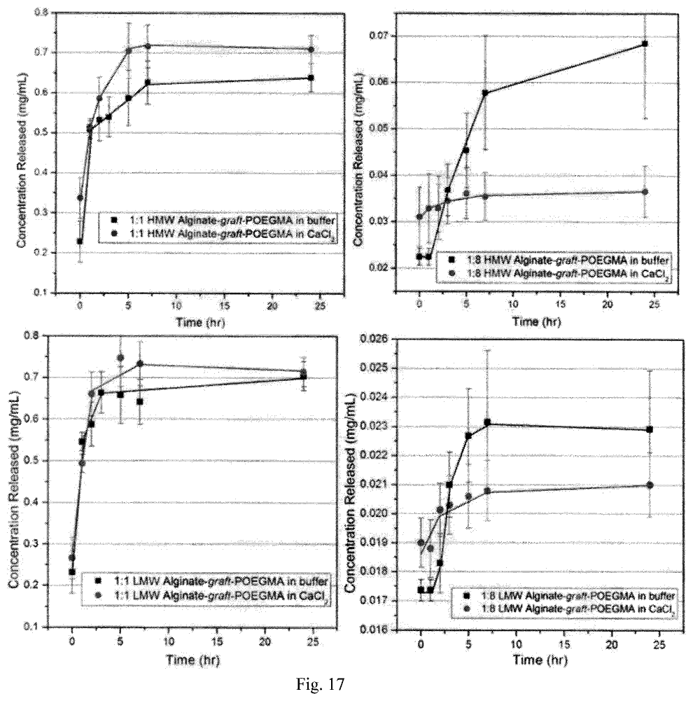

The percentage of encapsulation was the highest for the 1:2 ratio of polymer to 4BR, and the encapsulation efficiency remained fairly constant across the range. TEM images of the particles with a 1:1 polymer to 4BR ratio are shown in FIG. 16. In both cases, they appear denser than the unloaded particles, and show a 50% increase in size from 500 nm to 750 nm and 375 nm to 600 nm for the HMW and LMW samples, respectively. Centrifuged pellets of the nanoparticles with 1:1 and 1:8 ratios were introduced in equal amounts to solutions of sodium acetate buffer (pH=5) equilibrated at 37° C. and 2M CaCl₂ at 25° C. as part of the 4BR release study. Samples were taken at pre-determined intervals, centrifuged, and the supernatant was analysed for the concentration of 4BR by UV/Vis spectroscopy. The release of 4BR over time is reported in FIG. 17.

For the 1:1 samples, the release of 4BR does not seem to be influenced by the solution, as seen by similar release curves for particles in both solutions. The curves show an initial burst in 4BR release for the first 3 hours, followed by a stabilization of the total release thereafter. However, the 1:8 samples demonstrate a 4BR release profile that differs from the 1:1 samples. The initial concentration of 4BR in the supernatant is lower for samples in buffer as compared to those in CaCl₂. 4BR concentration begins to increase rapidly in buffer after an inhibition period of 1 hour, stabilizing at 4 hours. This is unlike the samples in CaCl₂, which only show a marginal increase of 4BR in the supernatant in the first 7 hours.

Encapsulation experiments were also performed by introducing CaCl₂ to solutions of 4BR and polymer in methanol, and these showed similarly high encapsulation efficiencies and loading percentages (Table 7)

TABLE 7
Encapsulation percentages for the various ratios
of 4BR to HMW and LMW alginate-graft-POEGMA when
CaCl₂ is introduced into the polymer solution

| | Ratio of | Encap | Loading |
|---|---|---|---|
| | 4BR:poly | (%) | (%) |
| HMW | 1:8 | 87 | 11 |
| | 1:4 | 68 | 17 |
| | 1:2 | 67 | 33 |
| | 1:1 | 64 | 64 |
| | 2:1 | 68 | 136 |
| LMW | 1:8 | 86 | 11 |
| | 1:4 | 84 | 21 |
| | 1:2 | 85 | 43 |
| | 1:1 | 87 | 87 |
| | 2:1 | 88 | 176 |

Particles of the same ratio (1:1 and 1:8) of 4BR to polymer were also dispersed in sodium acetate buffer at 37° C. Similar kinetics and total 4BR released were observed for all samples (FIG. 9a). This was unlike the release profiles for the previous method where the total quantity of 4BR released for the 1:1 samples was 8-10 times higher than that of the 1:8 samples with different release profiles across the samples. This result suggests that 4BR could be precipitating upon the addition of CaCl₂, particularly for the 1:1 sample, resulting in the inaccurate determination of encapsulation efficiencies. Due to this, the method of adding calcium to solutions of 4BR and the polymer may not be the most suitable method for the encapsulation of 4BR.

In summary, the release profile from the particles prepared by introducing alginate and 4BR into a solution of CaCl₂ suggest a high loading efficiency of 4BR with no indication of any calcium mediated 4BR precipitation outside of the nanoparticles. However, controlled release in an acidic medium was only realized for lower 4BR concentrations, and is presumably due to a high diffusive effect that is brought about by higher 4BR concentrations.

### EXAMPLE 14

### Encapsulation of Doxorubicin and Paclitaxel

Both doxorubicin (DOX) and paclitaxel (PTX) are commonly used chemotherapy drugs that are hydrophobic (Scheme 4). Thus, they are prime candidates for encapsulation systems. Alginate-graft-POEGMA is used to encapsulate these drugs in the same way that 4BR was encapsulated. The copolymer and DOX or PTX were dissolved in methanol at a 2:1 ratio, then added to a 2M CaCl₂ solution to form drug loaded alginate nanoparticles. TEM images (FIG. 20a and b) show dense DOX loaded nanoparticles with a size of approximately 200-300 nm

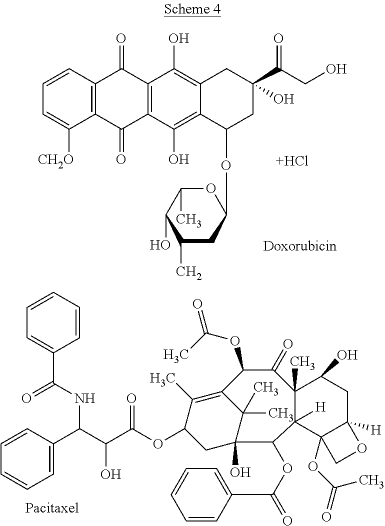

The supernatant calcium solutions from the drug loading were analyzed by UV/Vis to determine encapsulation efficiency, which ranged between 30%-60%.

### EXAMPLE 15

### Release of Doxorubicin

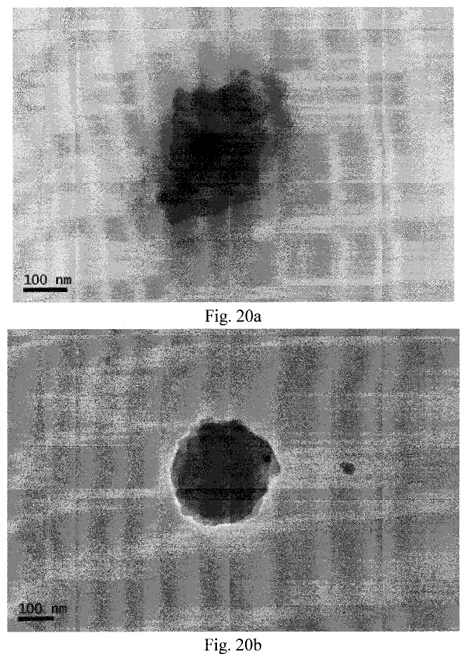

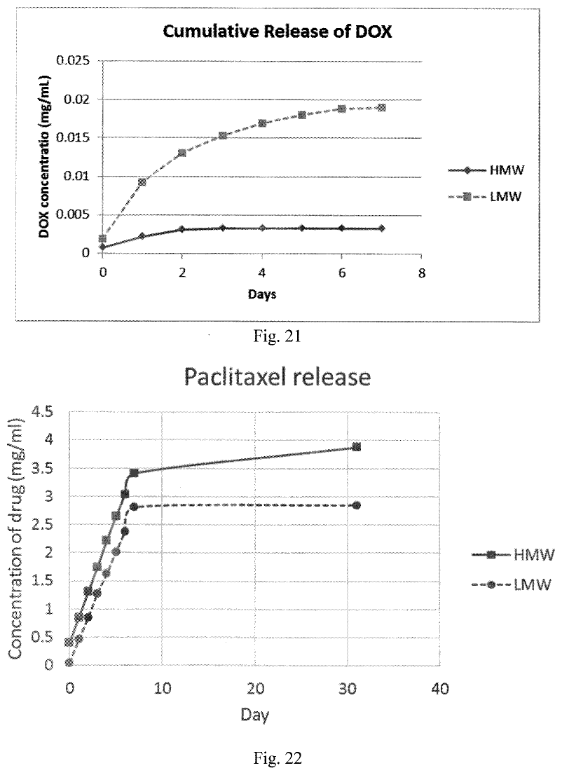

Doxorubicin loaded alginate nanoparticle solutions were prepared and 1 ml was placed in dialysis membrane tubes (MWCO 8-10k Da). Each of these dialysis tubes were placed 21 mL of sodium citrate buffer (pH 4.4) which was equilibrated at 37° C. The buffer was changed and stored daily, and at the end of 8 days, the stored buffer was analyzed by UV/Vis spectroscopy for the cumulative release of doxorubicin, shown in FIG. 21. The curve shows a gradual release over 8 days, with peak release observe only after the 7^{th} day for the LMW sample, and after the 4^{th} day for the HMW sample.

### EXAMPLE 16

### Release of Paclitaxel

Paclitaxel loaded alginate nanoparticles were dispersed in 10 mL of methanol/citrate buffer (10:90 v/v) and incubated at 37° C. Periodically, the suspensions were centrifuged and the entire supernatant was collected as a sample. Fresh solvent was replaced and the pellet was redispersed. At the end of a 30 day period, all samples were analyzed by UV/Vis to determine the cumulative drug release over time, and the data is plotted in FIG. 22. The data suggests an acid triggered release of the encapsulated drug over an extended period, and while slower than the release kinetics of 4BR and DOX, shows a similar overall trend.

### EXAMPLE 17

### Mucoadhesive Properties of Alginate-Graft-POEGMA

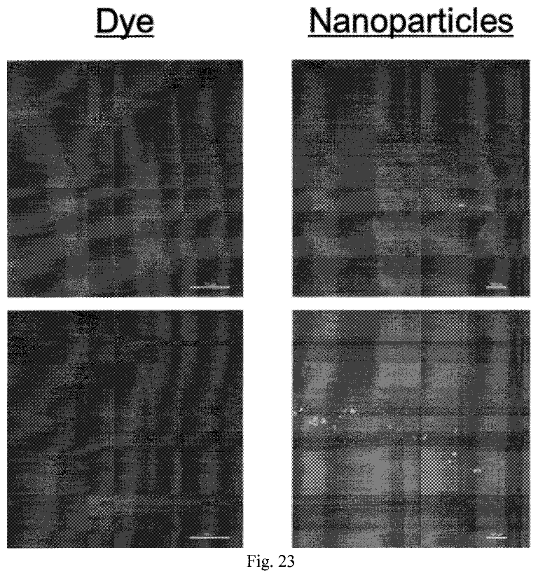

Alginate-graft-POEGMA nanoparticles were prepared by reaction with CaCl₂, and then labeled with a fluorescent dye. Samples of a pig's intestine were then washed separately with solutions of the fluorescent dye and fluorescent nanoparticles, then subsequently with deionized water. The fluorescence microscopy images are shown FIG. 23, it can be observed that intestine samples, that were washed with the nanoparticles, showed bright fluorescent spots, indicating that the fluorescent dye carrying nanoparticles had adhered to the intestinal tissue. Conversely, the intestine samples that were merely washed with the fluorescent dye did not show any change in fluorescence from an unwashed sample.

### EXAMPLE 18

### New Polymer—Alginate-Graft-poly(ethylene glycol)

A new copolymer, based on the previous synthesis protocol of BM1430-alginate was synthesized. Briefly, poly(ethylene) glycol (PEG) (Mₙ: 5000 Da) with an acid functional end-group was reacted with 1,1-carbonylimidazole in DMSO. The product solution was added to a solution of alginate-TBA in DMSO with 4% tetrabutylammonium fluoride and the mixture was left to react at 40° C. for 2 days. The product was purified by dialysis against deionized water, then analyzed by ¹H NMR to determine the degree of substitution. HMW and LMW alginate-graft-PEG had a degree of substitution of 0.39 and 0.265 respectively. The reaction pathway is shown in Scheme 5.

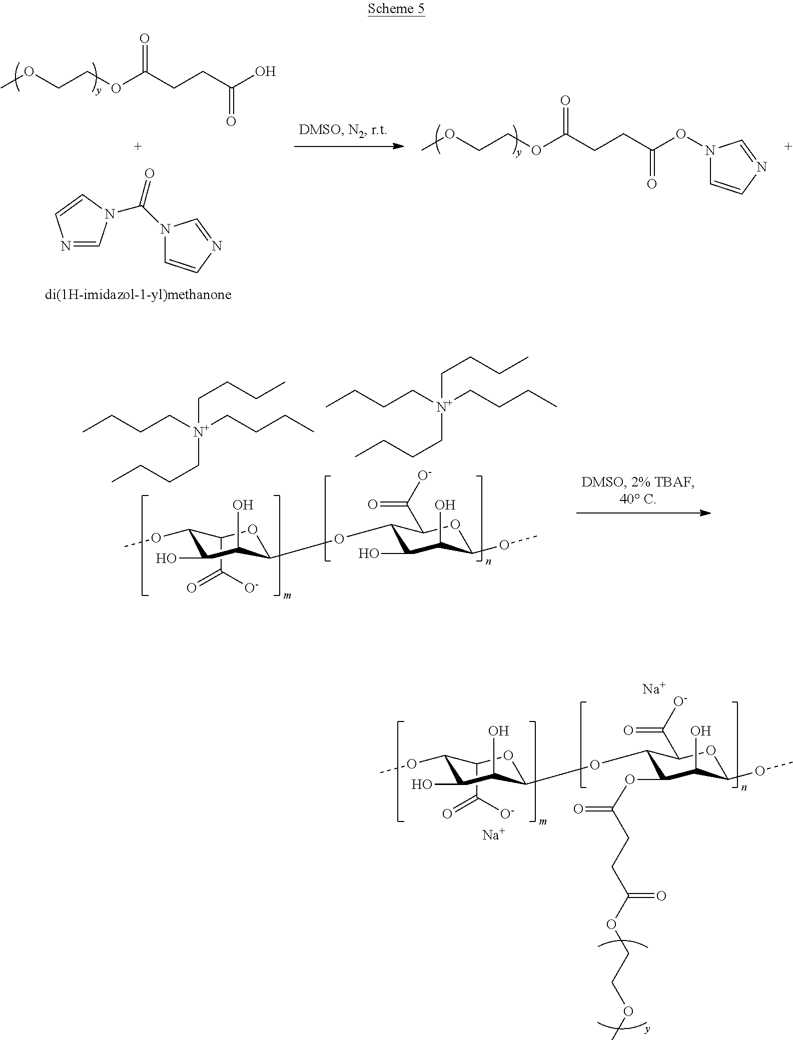

### EXAMPLE 19

### Ca²⁺ Response of Alginate-Graft-PEG

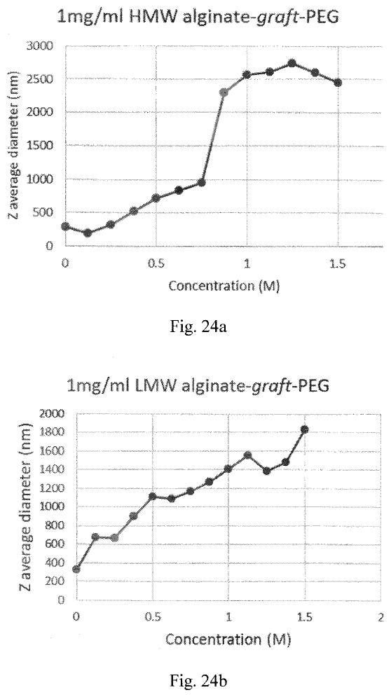

In solution, alginate-graft-PEG behaved similarly to alginate-graft-POEGMA when titrated against CaCl₂ when analyzed by dynamic light scattering. For the HMW sample, the same trend is observed where there is a marginal increase of particle size up til a point where there is a sudden and large increase of particle size. For the LMW sample, a similar trend is observed where the increase in particle size is more uniform and gradual. The response curves are shown in FIG. 24a and b.

### EXAMPLE 20

### Cisplatin Conjugation of Alginate-Graft-PEG

Cisplatin, a platinum complex with a 2+ charge, is a commonly used chemotherapy drug with poor water solubility. Since it is a divalent compound, it would serve as a cross-linker with alginate, much like Ca²⁺. Cisplatin was mixed with alginate-graft-PEG at a range of molar ratios of carboxylic acid moieties on the alginate backbone to platinum. The cross-linking reaction, which was performed at 40° C. over 2 days is illustrated in Scheme 6.

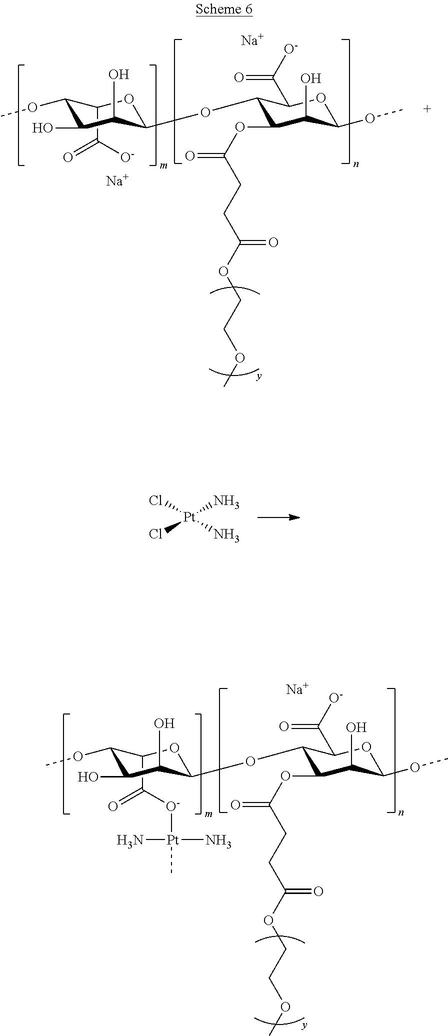

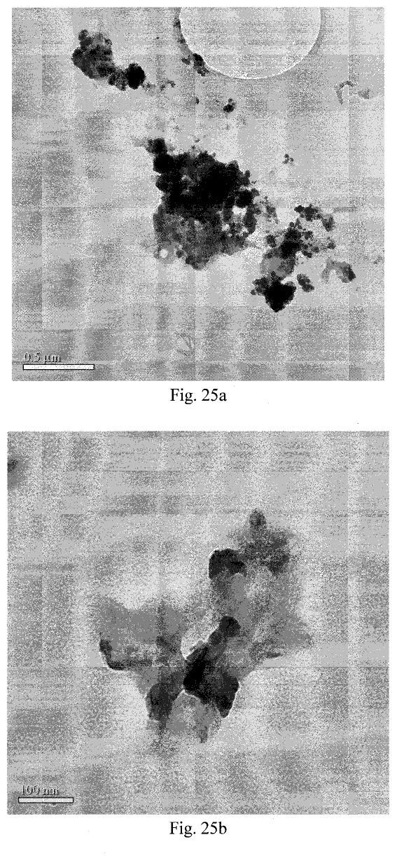

Dynamic light scattering of the polymer before and after reaction with cisplatin showed that the particle size increased remarkably for the HMW polymer and marginally for the LMW polymer, summarized in Table 8. TEM images (FIG. 25a and b), shows that the alginate-graft-PEG cross-linked with cisplatin takes on a more unstructured morphology. The platinum loading content was analyzed by thermogravimetric analysis (TGA), where the percent of platinum loaded in the nanoparticles was in a range between 5-40%.

TABLE 8
Summary of the particle size at different
cisplatin to acid moiety ratios

| | | z-avg size | |
|---|---|---|---|
| Polymer size | Pt/COOH | (nm) | PDI |
| HMW | 0 | 237.5 | 1 |
| | 0.25 | 2303 | 0.956 |
| | 0.50 | 583.7 | 0.581 |
| | 0.75 | 4299 | 0.543 |
| LMW | 0 | 109 | 0.585 |
| | 0.25 | 269.8 | 0.238 |
| | 0.50 | 177.6 | 0.256 |
| | 0.75 | 136.5 | 0.177 |

### EXAMPLE 21

### Release of Cisplatin

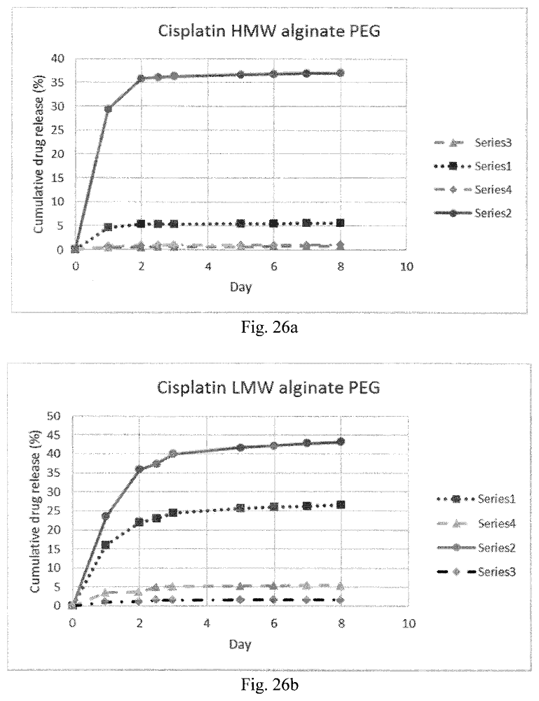

Solutions of cisplatin loaded polymeric nanoparticles which were prepared with in a range of 0.25 to 1 molar ratio of cisplatin to COOH moieties. A 1mL aliquot was taken and placed in a dialysis membrane (MWCO 8-10 kDa), which was immersed in 21 mL of phosphate buffered saline (0.15M NaCl) incubated at 37° C. The PBS aliquots were replaced and sampled at periodic intervals over 8 days. Each of the samples were analyzed by inductively couple plasma-mass spectrometry (ICP-MS) to analyse the concentration of platinum in the dialysate samples to obtain a cumulative release curve of platinum with time, shown in FIG. 26.

Conclusion. This body of work demonstrates the first example of polymer grafting on alginate via RAFT. Cations of both high molecular weight sodium alginate, and a depolymerized variant were first converted to tetrabutylammonium groups, which afforded solubility of the polymers in DMSO. A RAFT agent was functionalized on the hydroxyl moieties of both alginates in DMSO. POEGMA was polymerized from the alginate macroRAFT agents yielding comb copolymers of alginate-graft-POEGMA with intact carboxylic acid moieties on the alginate backbone. The polymerization from the macroRAFT agents demonstrated pseudo first-order rate kinetics, with a faster rate of polymerization for the smaller of the two macroRAFT agents. When calcium was titrated into solutions of alginate-graft-POEGMA, the polymers self-assembled into nanoparticles after a critical concentration of calcium (CCC) was reached. A direct correlation of polymer concentration to the critical calcium concentration was observed, while an inverse correlation was observed for polymer concentration to size and polydispersity of the assembled particles. There was observed that the polymer with a larger alginate backbone exhibited a more sudden size increase at the CCC while the smaller alginate backbone exhibited a more gradual assembly. Since self-assembly is driven by the rate of diffusion of Ca²⁺, which in turn would be influenced by polymer size, it seems likely that the more ordered self-assembly of the LMW polymers is a growth driven mechanism while the HMW self-assembly is heavily dependent on a critical point. Also, TEM images showed that the LMW particles adopted a classically spherical morphology while HMW particles had a varied and denser morphology. Self-assembly into nanoparticles was also shown in cases where the polymers were titrated into calcium solutions and acid was titrated into polymer solutions. Moreover, the polymers exhibited excellent solubility for a range of organic solvents, and similar calcium induced self-assembly characteristics were observed in methanol.

Due to the versatility of the polymers, they were exploited in the encapsulation of 4-n-butylresorcinol in methanol. This was performed by either: (1) adding calcium to a solution of the polymer and active; or (2) adding the polymer/4BR mixture to a solution of calcium. The materials demonstrated high encapsulation efficiency using both encapsulation techniques and two 4BR loading amounts were chosen for further analysis. Based on the release profiles of samples from method 1, a similar cumulative release of 4BR was observed for both loading quantities. The discrepancy in the calculated loading and released quantity could be attributed to 4BR that may have precipitated from solution upon the addition of calcium. For samples prepared by method 2, distinctly different release profiles were observed between higher and lower active concentrations, which potentially precludes any precipitation of 4BR. The release of 4BR at higher concentration occurred irrespective of the medium that the nanoparticles were in, but for lower concentrations of 4BR, the release was triggered, as expected, in mildly acidic buffer. These results imply that while these particles release their encapsulated payload at mildly acidic pH, there may be a point of overloading that could result in a loss of this phenomenon.

This body of work effectively demonstrates how alginate can be modified with hydrophilic brushes via RAFT polymerization and in doing so can afford self-assembly to nanoparticles upon the introduction of calcium ions. This surfactant-free method of preparing alginate nanoparticles was exploited in the encapsulation of a lipophilic drug with outstanding encapsulation efficiencies. The body of work shows tremendous potential for the use of biodegradable and sustainable materials for drug delivery.

1. A modified alginate copolymer comprising an alginate backbone and having a grafted moiety attached to one of the hydroxyl groups of the alginate backbone, the grafted moiety comprising a polymer and a stabilizing group, the stabilizing group comprising at least 2 heteroatoms independently selected from the group consisting of N, S, P and Si.

2. The modified alginate copolymer of claim 1, wherein the polymer is an acrylate-based polymer.

3. The modified alginate copolymer of claim 2, wherein the acrylate-based polymer is a methacrylate-based polymer.

4. The modified alginate copolymer of claim 1, wherein the polymer comprises an oligo(ethylene glycol) moiety.

5. The modified alginate copolymer of claim 4, wherein the oligo(ethylene glycol) moiety is attached to the oxygen atom of a carbonyl ester.

6. The modified alginate copolymer of claim 1, wherein the grafted moiety is attached to one of the hydroxyl groups of the alginate backbone by way of a carbonyl ester bond.

7. The modified alginate copolymer of claim 1, wherein the stabilizing group is a functional group selected from the group consisting of a thiocarbonate, an azide and a 5 or 6-membered heterocycle.

8. The modified alginate copolymer of claim 7, wherein the thiocarbonate is a trithiocarbonate.

9. The modified alginate copolymer of claim 7, wherein the 5 or 6-membered heterocycle is an azole or azoline.

10. The modified alginate copolymer of claim 9, wherein the azole is selected from the group consisting of pyrazole, triazole, imidazole, 1-pyrazoline, 2-pyrazoline, 3-pyrazoline, 1, 2, 3-thiadiazole, 1, 2, 4-thiadiazole, 1, 2, 5-thiadiazole, 1, 3, 4-thiadiazole, 1, 4, 2-dithiazole, 1, 2, 5 dithiazole, 1, 3, 4 dithiazole and 1, 2, 2, 4-dithiazole.

11. The modified alginate copolymer of claim 1, wherein the alginate backbone has a molecular weight between 1 and 1000 kDa.

12. The modified alginate copolymer of claim 1, wherein the alginate backbone has been modified to have a molecular weight between 10 and 500 kDa.

13. A drug delivery method, the method comprising dissolving the modified alginate copolymer of claim 1 in a first solvent, and subjecting the solution to an M²⁺-containing source, subsequently collecting the solid of the ensuing reaction mixture and redispersing the obtained solid in a second solvent.

14. The drug delivery method of claim 13, wherein the method further comprises subjecting the solution to a bioactive agent before subjection to the M²⁺-containing source.

15. The drug delivery method of claim 13, wherein the M²⁺-containing source is selected from the group consisting of a Ca²⁺-containing source and a Pt²⁺-containing source.

16. (canceled)

17. The drug delivery method of claim 15, wherein the M²⁺-containing source is an aqueous solution comprising CaCl₂ and/or Pt(NH₃)₂Cl₂.

18. (canceled)

19. A process for making a modified alginate copolymer of claim 1 comprising subjecting alginate to an acid to obtain alginic acid, subjecting alginic acid to an alkylammonium solution to obtain an aginate-alkylammonium-salt, grafting a moiety on the alginate backbone, the moiety comprising a stabilizing group, and polymerizing the grafted moiety with a polymerizable moiety, wherein one of the grafted moiety or the polymerizable moiety comprises a stabilizing group, the stabilizing group comprising at least 2 heteroatoms independently selected from the group consisting of N, S, P and Si.

20-26. (canceled)

27. An alginate nanoparticle comprising a modified alginate copolymer of claim 1 and M²⁺.

28. (canceled)

29. The alginate nanoparticle of claim 27, wherein the alginate nanoparticle encapsulates the M²⁺.

30-35. (canceled)

36. A method of treating cancer comprising administering an effective amount of the alginate nanoparticle of claim 29 to a mammal, wherein the alginate nanoparticle further comprises a bioactive agent.

37. (canceled)
